(19) United States
(12) Patent Application Publication
(10) Pub. No.: US 2026/0080531 A1
(43) Pub. Date: Mar. 19, 2026

(54) SYSTEMS, METHODS AND DEVICE FOR SCREENING AND DIAGNOSIS OF CARDIOVASCULAR DISEASE

(71) Applicant: Yanran Wang, Santa Clara, CA (US)

(72) Inventor: Yanran Wang, Santa Clara, CA (US)

(21) Appl. No.: 19/204,333

(22) Filed: May 9, 2025

Related U.S. Application Data
(60) Provisional application No. 63/645,683, filed on May 10, 2024.

(51) Int. Cl. G06T 7/00; A61B 5/00; A61B 5/055; G06V 10/764; G06V 10/82; G16H 50/20

(52) U.S. Cl. CPC G06T 7/0012; A61B 5/0044; A61B 5/055; A61B 5/7264; A61B 5/7275; G06V 10/764; G06V 10/82; G16H 50/20; G06T 2207/10016; G06T 2207/10088; G06T 2207/10116; G06T 2207/20081; G06T 2207/20084; G06T 2207/30048; G06T 2207/30096; G06T 2207/30101; G06V 2201/031

(57) ABSTRACT

Disclosed are methods, systems, and a device for automated interpretation of cardiac magnetic resonance imaging. A method includes acquiring a sequence of radiographic images of a heart, including at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence. Further, the method processes the sequence of radiographic images using one or more machine learning models. Additionally, the method generates a diagnostic prediction using the machine learning models. The diagnostic prediction is a screening prediction of a cardiac anatomy, a diagnostic suggestion of a cardiovascular condition, a quantitative assessment of a cardiac function parameter, a structured radiographic report, a natural language summary, or a diagnostic rationale. A diagnostic prediction is output to an electronic system.

### CROSS REFERENCE TO RELATED APPLICATION

This application claims benefit of U.S. Provisional Application Ser. No. 63/645,683 filed May 10, 2024, the entire disclosure of which is incorporated herein by reference.

### FIELD OF THE DISCLOSURE

The present disclosure is generally related to methods and devices for medical imaging analysis, and more particularly is related to a system, methods, and device for screening and diagnosis of cardiovascular disease.

### BACKGROUND OF THE DISCLOSURE

Cardiovascular diseases (CVDs) are the number one leading cause of death in the world. According to the World Health Organization, an estimated 17.9 million people die each year from CVDs, accounting for approximately 32% of all deaths worldwide. Among these, over 75% of CVD deaths occur in low—and middle-income countries. The most widely used screening exams for CVDs-electrocardiogram (ECG) and echocardiogram (echo)—capture only a fraction of the informative features for diagnosis. Additionally, some conventional diagnostic techniques for CVD are invasive and may lead to side effects. For example, diagnosis of Pulmonary Arterial Hypertension (PAH) is right heart catheterization (RHC), which is an invasive procedure that can introduce serious surgical complications including hematoma, pneumothorax, arrhythmias, and hypotensive episodes. Although multiple approaches can be used to diagnose CVDs, cardiac magnetic resonance imaging (CMR) is a comprehensive imaging modality well suited to evaluate cardiac morphology, function, myocardial perfusion, and unique tissue characterization. However, widespread clinical implementation of CMR has been hindered by the time cost of CMR interpretation, considerable training time and efforts to gain the expertise, and the resulting shortage of qualified CMR-trained doctors. The limited availability of adequately trained CMR experts can make timely and accurate diagnosis of CVD using CMR extremely difficult, costly, time-consuming, and susceptible to operator bias.

As shown in FIG. 1, a typical CMR exam may have short-axis cine films with 9 parallel views (typical 25 frames/view), a four-chamber cine film (25 frames), a three-chamber cine film (25 frames), short-axis LGEs (9 parallel views), and four-chamber LGE, leading to at least videos and 10 images to analyze in total. (FIG. 1, steps 20 and 40). The standard clinical approach to CMR interpretation requires experts to (1) manually delineate the contours of the endocardium and epicardium, and (2) scan back and forth across cine film and LGE over a series of short-axis and long-axis views before proposing a diagnosis. (FIG. 1, steps 60 and 70).

Hence, this procedure is extremely labor-intensive, time-consuming, and susceptible to operator bias. A significant global shortage of physicians trained in cardiac magnetic resonance (CMR) imaging presents a critical barrier to timely and accurate cardiovascular diagnosis. Additionally, conventional practice leads to the patient spending extended periods in the MRI machine and invasive exposure to the contrast agent to acquire the views. Some patients, such as pediatric patients, those with claustrophobia, and those with allergies to contrast agents, cannot tolerate long time periods in an MRI or the contrast injection required to collect all of the views in conventional CMR. (FIG. 1 steps 20 to 50)

Thus, a heretofore unaddressed need exists in the industry to address the aforementioned deficiencies and inadequacies.

### SUMMARY OF THE DISCLOSURE

Embodiments of the present disclosure provide systems, methods, and a device for screening and diagnosis of cardiovascular disease. Briefly described, one embodiment of the method, among others, can be described as a computer-implemented method for automated interpretation of cardiac magnetic resonance (CMR) imaging. In this regard, one method, among others, can be broadly summarized by the following steps: acquiring a sequence of radiographic images of a cardiovascular system, where the sequence of radiographic images is at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence; processing the sequence of radiographic images using one or more machine learning models, wherein at least one of the machine learning models is a deep learning model; generating a diagnostic prediction using the one or more machine learning models, wherein the diagnostic prediction is at least one of: a screening assessment of a cardiac anatomy, a diagnostic identification of a cardiovascular condition, a quantitative evaluation of a cardiac function parameter, a structured radiographic report, a natural language summary, or a diagnostic rationale; and outputting the diagnostic prediction to at least one of an electronic user interface, an electronic health record system, a cloud-based platform, a mobile application, or a picture archiving communication system.

Another embodiment can be described as a computer-implemented method for automated diagnosis of cardiovascular diseases using a two-stage deep learning pipeline. One method, among others, can be broadly summarized by the following steps: acquiring a cine MRI sequence of radiographic images of a cardiovascular system without a contrast agent; processing the cine MRI sequence of radiographic images using one or more machine learning models, wherein at least one of the one or more machine learning models is at least one of a video-based transformer, a convolutional neural network, a recurrent neural network, a transformer-based model, or a multi-modal hybrid architecture; detecting at least one of a cardiac anomaly, anatomical variation, or a functional abnormality using the cine MRI sequence of radiographic images in a first stage; and generating a diagnostic classification using the cine MRI sequence of radiographic images and at least one of a late gadolinium enhancement MRI, a T1 mapping, a T2 mapping, a perfusion imaging, a flow quantification, a dark blood imaging, a real-time imaging, a magnetic resonance spectroscopy, or a parametric mapping sequence, in a second stage.

Yet another embodiment of the present disclosure provides a computerized system for automated interpretation of cardiac magnetic resonance imaging. Briefly described, in architecture, one embodiment of the system, among others, can be implemented as follows. A computerized system for automated interpretation of cardiac magnetic resonance imaging has a computerized device has a non-transitory memory, one or more processing apparatuses in communication with the non-transitory memory, and a computer readable storage medium. A magnetic resonance imaging (MRI) machine is in communication with the computerized device. The MRI machine is configured to acquire a sequence of radiographic images of a cardiovascular system, wherein the sequence of radiographic images is at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence. One or more programs comprising program instructions is stored on the computer readable storage medium and is executable by the one or more processing apparatus via the non-transitory memory. The instructions comprise: processing the sequence of radiographic images using one or more machine learning models, wherein at least one of the one or more machine learning models is a video-based transformer model, a convolutional neural network (CNN), a recurrent neural network (RNN), a deep neural network (DNN), a graph-based neural network, or a model capable of processing at least one of sequential data or spatiotemporal data; generating a diagnostic prediction using the one or more machine learning models, wherein the diagnostic prediction is at least one of a screening assessment of a cardiac anatomy, a diagnostic identification of a cardiovascular condition, a quantitative evaluation of a cardiac function parameter, a structured radiographic report, a natural language summary, or a diagnostic rationale; and outputting the diagnostic prediction to at least one of an electronic user interface, a picture archiving and communication system, a cloud-based platform, a mobile application, or an electronic health record.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a diagrammatical flowchart illustrating conventional CMR in accordance with the prior art.

FIGS. 2A and 2B are diagrammatical flowcharts illustrating a screening and diagnosis method, in accordance with the present disclosure.

FIG. 3 is a diagrammatical flowchart illustrating a method for screening and diagnosis of cardiovascular diseases in accordance with the present disclosure.

FIG. 4 is a diagrammatical flowchart illustrating a method for automated interpretation of cardiac magnetic resonance (CMR) imaging in accordance with the present disclosure.

FIG. 5 is an illustration of a deployment of a screening and diagnostic systems, methods, and device in accordance with the present disclosure.

FIG. 6 is a diagrammatical flowchart illustrating a computer-implemented method for analysis of cardiac magnetic resonance imaging in accordance with the present disclosure.

FIGS. 7A and 7B are flowcharts showing an inclusion-exclusion cascade of the screening and diagnostic systems, methods, and device in accordance with the present disclosure.

FIG. 8 is an illustration of cardiac MRIs utilized in model development of the screening and diagnostic systems, methods, and device in accordance with the present disclosure.

FIGS. 9A-9D are diagrammatical illustrations of performance curves characterizing the screening and diagnosis systems, methods and device, in accordance with the present disclosure.

FIGS. 10A-10B are diagrammatical illustrations depicting additional characterizations of the screening and diagnostic methods, systems, and device in accordance with the present disclosure.

FIG. 11 is a series of images showing characterizations of the screening and diagnostic methods, systems, and device in accordance with the present disclosure.

FIGS. 12A and 12B show a schematic overview of a portion of a model used in the screening and diagnostic systems, methods and device, in accordance with the present disclosure.

FIGS. 13A-13C show distributions of a characteristic of the screening and diagnostic systems, methods, and device in accordance with the present disclosure.

FIGS. 14A and 14B show clinical prevalence of CVD classes in accordance with the present disclosure.

FIGS. 15A and 15B show the preprocessing step for the screening and diagnostic systems, methods, and device in accordance with the present disclosure.

FIG. 16 shows characteristics of learning rate modification of the screening and diagnostic systems, methods, and device in accordance with the present disclosure.

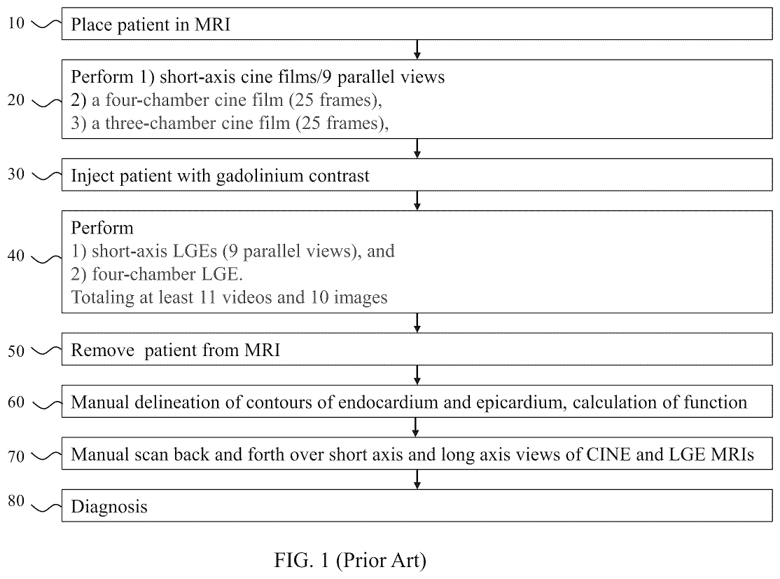

### DETAILED DESCRIPTION

The computer-implemented method for screening and diagnosis of cardiovascular disease may include screening for cardiac anomalies using nonenhanced cine magnetic resonance imaging (MRI) followed by diagnosing cardiovascular diseases using cine and late gadolinium enhancement (LGE) MRI as combined inputs. As used throughout, it is understood that late gadolinium enhancement (LGE) may substitute for any enhancement MRI using any type or composition of contrast agent.

Example systems and methods for automated and interpretable analysis of cardiac magnetic resonance (CMR) imaging may use artificial intelligence. The disclosure may enable a fully automated, clinically relevant pipeline that performs screening, diagnosis, cardiac function quantification, and comprehensive radiographic reporting by leveraging deep learning models. In an example, the system and method may use two or more serial video-based swin transformer (VST)-based AI models, which may include a screening model and a diagnostic model.

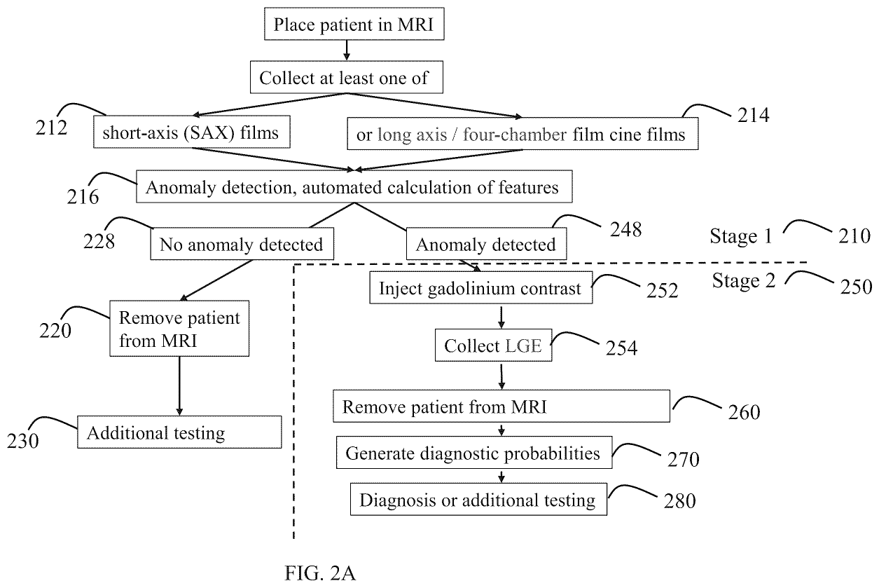

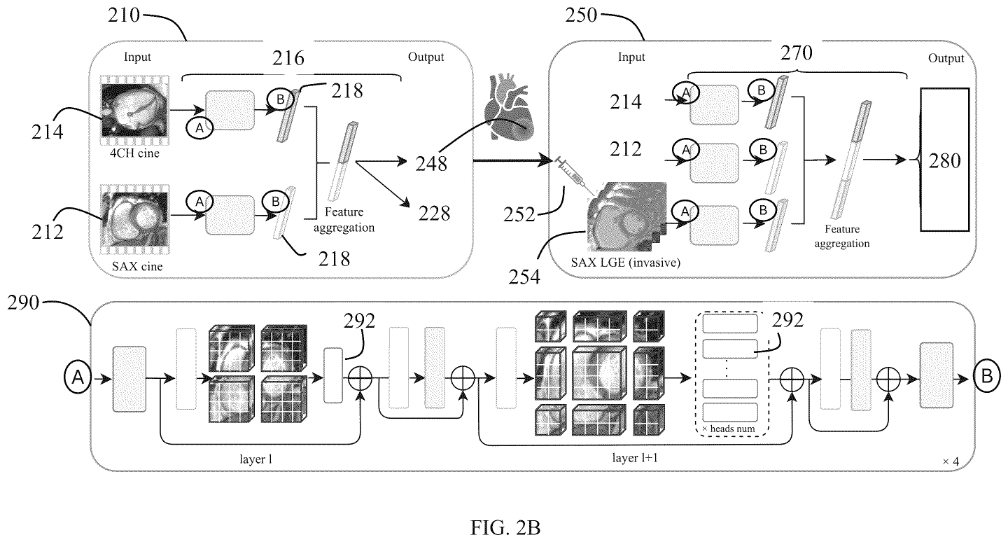

FIGS. 2A and 2B are diagrammatical flowcharts illustrating an example of a screening and diagnosis method, in accordance with examples of the present disclosure. Referencing FIGS. 2A and 2B may help to understand a deep learning approach for automatic/computerized CMR interpretation and diagnosis which may have a two-stage paradigm. FIG. 2A shows an example of a two-stage deep learning system which may include a first stage 210 that performs anomaly screening 216 using non-contrast cine MRI 212 and/or 214. The cine MRI may use 3 parallel views. A four chamber can use one or more views. T1, T2, and other mapping sequences May also be used. An anomaly 248 may be detected or an anomaly may not be detected 228 in the first stage 210 and based on that result, the patient may be removed from the MRI 220 or May proceed to a second stage 250. The first stage 210 may be followed by a second stage 250 that conducts diagnostic classification using cine MRI 212 and/or 214 in combination with contrast-enhanced sequences, such as late gadolinium enhancement (LGE) 254.

The initial stage based on cine modality may enable a noninvasive cardiac screening. Compared to LGE, which requires the injection of a gadolinium 252 or other contrast agent, cine MRI may be safer and more easily acquired. For example, avoiding gadolinium contrast may be safer for pediatric patients and those who are intolerant of or allergic to the contrast through avoidance of side effects of contrast injection and skin puncture to inject the contrast. Though a patient may be able to avoid side effects from allergies or intolerance to one type of contrast by selecting another contrast, the patient still runs a risk of side effects from the invasive nature of the contrast injection. Thus, no matter what contrast agent is used, the patient's safety may be enhanced by avoiding injection of any contrast agent.

Additionally, for patients who cannot tolerate long periods in an MRI, such as pediatric patients or those with claustrophobia, enabling a diagnosis while avoiding the LGE scans or otherwise reducing the number of scans required in comparison to conventional CMR may be beneficial by reducing their time required in the MRI. The systems and methods illustrated in this disclosure may support both sequential and unified multitask model architectures and May operate in a cine-only mode, enabling contrast-free diagnosis. Enabling contrast-free diagnosis may be particularly advantageous in low-resource settings (for example, where MRI time is limited or gadolinium is unavailable) or patients who cannot tolerate contrast injection.

The second stage 250 may provide classification 270 of eleven types of CVDs covering most patients referred to the CMR examination, which may include ischemic heart disease, most types of nonischemic cardiomyopathy, pulmonary hypertension, and congenital heart disease. Table 1 lists eleven types of CVDs. The classification may be provided as a suggestion of a diagnosis or a diagnostic suggestion of a cardiovascular condition.

Stage one 210 may include screening for anomalies 248 using nonenhanced cine MRI 212 and 214, which may be followed by stage two 250, which may include diagnosing cardiovascular diseases 280 using SAX cine 212 and/or 4CH cine 214 and late gadolinium enhancement (LGE) MRI 254 as combined inputs. FIGS. 2A and 2B may represent a workflow of the two-stage paradigm for automatic screening and diagnosis of cardiovascular diseases. In FIGS. 2A and 2B and throughout, SAX stands for short axis; 4CH stands for four-chamber; and MLP stands for multilayer perceptron.

FIG. 2B is an illustration of an automatic pipeline in accordance with this disclosure. The automatic pipeline may have two serial VST-based AI models: the screening model 216 and the diagnostic model 270. A VST may also refer to a feature encoder. Referring to FIG. 2B, for each patient, the screening model 216 may take cine movies 212 and 214 as inputs at A, and outputs at B the binary classification 218 to detect cardiac anomaly 248. The binary classifications 218 May be features that may be aggregated to assist the detection of the anomaly 248 as part of model 216. An anomaly 248 may also be referred to as an abnormality. The initial stage 210 based on cine modality 214 and/or 212 may enable a noninvasive cardiac screening. If no anomaly or normal 228 is detected in stage one 210, a patient may be removed from the MRI 220 and not subjected to the gadolinium contrast injection 252. The patient may be able to receive a diagnosis 230 from the stage one imaging 210 or may go on to additional testing for diagnosis 230. A benefit of the two-stage approach in FIGS. 2A and 2B is that it may enable a patient to completely avoid a gadolinium contrast injection 252 or being exposed to any contrast agent. The patient suspected of cardiac anomaly 248 may undergo LGE imaging 254 in stage two 250. The diagnostic model 270 may integrate both cine 212 and/or 214 and LGE 254 to output their CVD class 280.

Further, this disclosure may demonstrate which imaging modality (cine 212 and 214 or LGE 254), view (four-chamber 214 or short-axis 212), and their aggregation may be utilized for optimal classification performance. This disclosure may create an avenue for accurate CMR interpretation in real-time or near real-time, and may encourage more widespread use of CMR in CVD screening and diagnosis.

A video-based swin transformer (VST) 290 may be a preferred model backbone, instead of the conventional convolutional neural network (CNN) approach, because of the superiority of the transformer model in modelizing CMR sequences. The diagnostic model 270 may integrate both cine 212 and 214 and LGE 254 to output their CVD class 280. The AI models May comprise four video-based swin transformant (VST) blocks 290 to analyze the CMR sequences using 3D shifted window self-attention (WSA) mechanism 292. Transformer-based deep learning architectures may yield significant improvements on a wide spectrum of high-level computer vision tasks. VST is a transformer adapted for video sequence processing with impressive performance on the major video recognition benchmarks. However, few efforts have been made to explore its role in medical video analysis. As opposed to the conventional CNNs, which are limited by the small receptive field of the convolution operation, the global self-attention and shifted window mechanism which may be inherent in VST broadens the receptive field and allows effective integration of temporal and spatial information from cardiac video and 3D sequences, in a way not readily achievable by the human mind. The superior performance of VST over CNN is demonstrated in this disclosure.

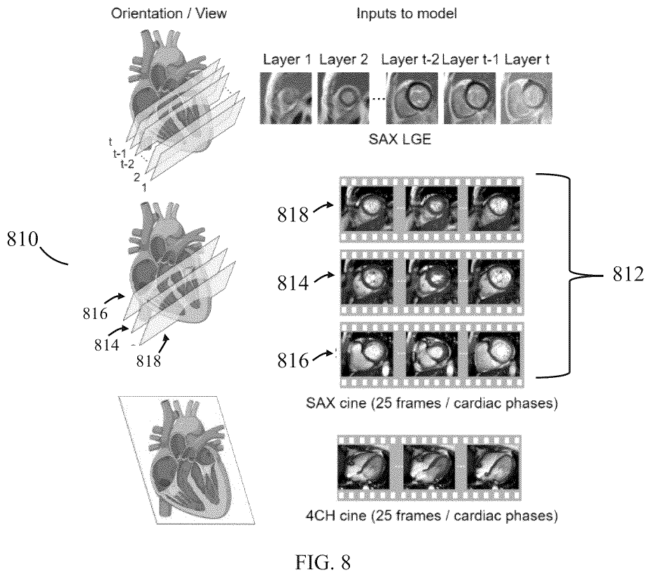

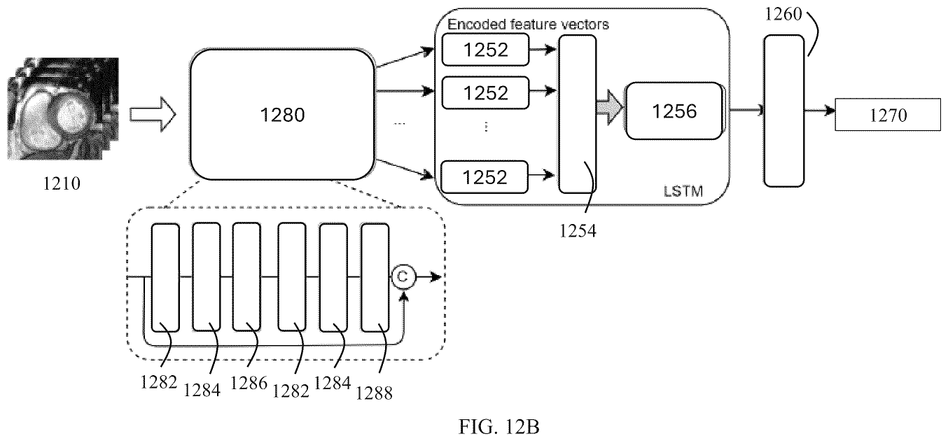

The AI models may include four video-based swin transformer (VST) blocks 290 to analyze the CMR sequences 212, 214, and 254 using 3D shifted window self-attention (WSA) mechanism 292. The sequences may include SAX, or short-axis; 4CH, or four-chamber; MLP, or multilayer perceptron. The image sequences may be understood with reference to FIG. 8, described later herein. VST block 290 may be additionally understood by reference to FIG. 12A, and particularly block 1290, more fully described later in this disclosure.

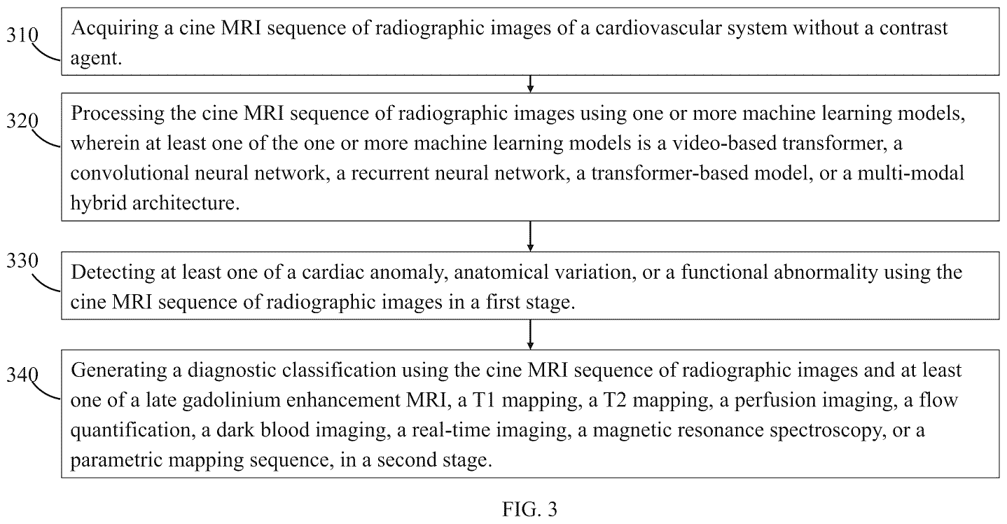

FIG. 3 is a flowchart illustrating a computer-implemented method for automated diagnosis of cardiovascular diseases using a two-stage deep learning pipeline in accordance with an example of the disclosure. It should be noted that any process descriptions or blocks in flow charts should be understood as representing modules, segments, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

Step 310 includes acquiring a cine MRI sequence of radiographic images of a cardiovascular system without a contrast agent.

Step 320 includes processing the cine MRI sequence of radiographic images using one or more machine learning models, wherein at least one of the one or more machine learning models is at least one of a video-based transformer, a convolutional neural network (CNN), a recurrent neural network (RNN), a transformer-based model, or a multi-modal hybrid architecture.

Step 330 includes detecting at least one of a cardiac anomaly, an anatomical variation, or a functional abnormality using the cine MRI sequence of radiographic images in a first stage. The cine MRI sequence may be without enhancement or a contract agent. The first stage may be based solely on the cine MRI sequence of radiographic images.

Step 340 includes generating a diagnostic classification using the cine MRI sequence of radiographic images and at least one of a late gadolinium enhancement MRI, a T1 mapping, a T2 mapping, or a parametric mapping sequence in a second stage. Generating may include additional imaging modalities.

Any number of additional steps, functions, processes, or variants thereof may be included in the method, including any disclosed relative to any other figure of this disclosure.

As an example, at least one of the one or more machine learning models is a video-based swin transformer.

In another example, a multi-stage pipeline may be configured to operate in a cascading or parallel processing manner. A diagnostic accuracy may be enhanced through iterative refinement and multi-view integration. In another example, the method may further comprise generating a quantitative cardiovascular function metric, a structured radiographic report, an interpretability visualization, and a diagnostic rationale based on a detected anomaly or classification, which may also support downstream clinical decision-making.

In an example in accordance with this disclosure, a method may include selecting a treatment based on the diagnostic classification. In another example of a method in accordance with the present disclosure, the cine MRI sequence of radiographic images may be at least one of: a short-axis view, a long-axis view, or a selected representative slice. In another example in accordance with this disclosure, the method may include diagnosing one or more cardiovascular diseases using CMR without injecting a contrast agent into a patient.

Further examples in accordance with the present disclosure may include particular imaging modalities (e.g., cine or LGE), views (e.g., four-chamber or short-axis), and their aggregation, which may be utilized for optimal classification performance. The method May create an avenue for accurate CMR interpretation in real-time, as well as bringing CMR into more widespread use in CVD screening and diagnosis.

An example in accordance with this disclosure may further enable cardiac function analysis through deep learning modeling of CMR sequences. The deep learning modeling May provide quantitative cardiac measurements such as left ventricular ejection fraction, right ventricle ejection fraction, a wall thickness, a cardiac output, an end-diastolic volume, a systolic volume, a stroke volume, a wall motion index, a myocardial strain, a myocardial perfusion, a tissue characterization, a right ventricular volume, a left ventricular volume, a cardiac workload, a myocardial workload, a ventricular mass, a left atrial volume, a right atrial volume, or a left ventricular outflow tract velocity. Model outputs may include both structured radiographic reports and natural language summaries generated via integrated large language models conditioned on vision module or imaging features.

The ability of deep learning to learn distinctive features and recognize motion patterns from raw input images and videos without requiring hand-crafted feature engineering and extensive data preprocessing may make it highly effective for interpreting CMR data. Furthermore, deep learning algorithms may have a clear advantage over humans by analyzing all images and dynamic pieces of information simultaneously and uniformly, offering more efficient and objective or non-biased solutions. The few applications of deep learning in CMR to date have focused on single aspects of CMR interpretation (e.g., limited to segmentation or wall thickness measurement) or have demonstrated limited diagnostic capabilities (e.g., limited to myocardial scarring or aortic valve malformations).

A video-based swin transformer (VST)—a cutting-edge advancement in computer vision—may be used as a model backbone of choice, instead of the conventional convolutional neural network (CNN) approach. The VST model may be superior to CNN in modelizing CMR sequences. Sequential or spatiotemporal data may be processed by a deep learning model or architecture.

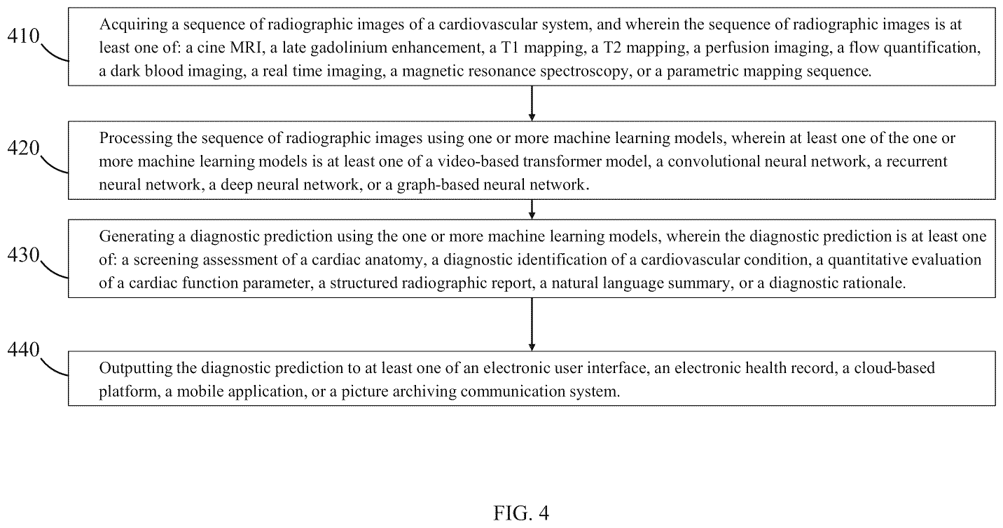

FIG. 4 is a flowchart illustrating a method for a computer-implemented method for automated interpretation of cardiac magnetic resonance (CMR) imaging in accordance with the disclosure.

Step 410 includes acquiring a sequence of radiographic images of a cardiovascular system, wherein the sequence of radiographic images is at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence.

A flow quantification may include, for example, 4D flow MRI. The imaging modality or sequence may provide relevant structural or functional information of the cardiovascular system.

Step 420 includes processing the sequence of radiographic images using one or more machine learning models, wherein at least one of the one or more machine learning models is at least one of a video-based transformer model, a convolutional neural network, a transformer-based model, a CNN-transformer model, a CNN-transformer hybrid model, a vision-language hybrid model, a large language model, a recurrent neural network, a generative adversarial network, a deep neural network, a graph neural network, a multi-modal model, a self-supervised learning model, a semi-supervised learning framework, an attention-based model, a reinforcement learning model, or a model that integrates patient data with imaging data.

The one or more machine learning models may have an architecture that processes sequential or spatiotemporal data.

Step 430 includes generating a diagnostic prediction using the one or more machine learning models, wherein the diagnostic prediction is at least one of: a screening assessment of a cardiac anatomy, a diagnostic identification of a cardiovascular condition, a quantitative evaluation of a cardiac function parameter, a structured radiographic report, a natural language summary, or a diagnostic rationale.

In an example, the natural language summary may have a finding. A finding may include an interpretation of or observation relating to the structure of a heart, for example. The finding may include identification of a cardiac anomaly or a normal structure.

In another example, the natural language summary may have a piece of information derived from the radiographic image sequence.

In another example, the diagnostic rationale may include a differential diagnosis and an explanation of a reasoning process. In a further example, the diagnostic rationale may also have an exclusion of a condition. The differential diagnosis may identify a disease or condition from a set of possible alternatives that could be causing a patient's symptoms, systematically distinguish between two or more conditions that present with similar clinical symptoms, and/or rule out less likely conditions and narrow down to the most probable diagnosis.

In another example, the diagnostic rationale may be at least one of a visual overlay, a textual explanation, or an interactive interpretability feature, a visual map highlighting a relevant region of the radiographic image sequence, an image overlay, a segmentation mask, a saliency map, an attention-based visualization, a textual explanation derived from a model parameter or a model activation, a natural language justification generated using a language model, a piece of evidence derived from the radiographic image sequence, a cardiac function, an exclusion of an alternative condition, a confidence score, a cardiac function assessment, or a clinical pathway suggestion.

Step 440 includes outputting the diagnostic prediction to at least one of: an electronic user interface, an electronic health record, a cloud-based platform, a mobile application, or a picture archiving communication system.

The diagnostic prediction may be output to a computing system capable of displaying or storing medical data. Any number of additional steps, functions, processes, or variants thereof may be included in the method, including any disclosed relative to any other figure of this disclosure.

In another example in accordance with this disclosure, the method may include selecting, recommending, or suggesting a treatment, intervention, follow-up examination, or follow-up actions based on at least one of the diagnostic prediction or the diagnostic rationale.

In another example in accordance with this disclosure, a method may additionally include extracting a heart region from the sequence of radiographic images.

In yet another example in accordance with this disclosure, a method may also have the diagnostic prediction further including a classification of the cardiovascular condition, wherein the cardiovascular condition is at least one of: an ischemic heart disease, a nonischemic cardiomyopathy, a pulmonary hypertension, a congenital heart disease, a valvular heart disease, a pericardial disease, an aortic disease, a heart failure syndrome, a myocardial abnormality, an endocardial abnormality, a rhythm disorder, a rare cardiovascular condition, and a post-treatment cardiac condition. Additionally, the rare cardiovascular condition may be at least one of a cardiac tumor, a congenital coronary anomaly, a fabry disease, or a marfan syndrome-related cardiac involvement.

In a further example, generating the diagnostic prediction using the one or more machine learning models may include sequentially generating the screening prediction of the cardiac anatomy and the classification of the cardiovascular condition, and wherein the one or more machine learning models is at least one of: a single multitask neural network architecture, a cascading output model, a hybrid model, or an ensemble model. A hybrid model may combine the one or more machine learning models. For example, a hybrid model may combine CNN and transformer architectures. In another example, a vision-language hybrid model may process both visual and textual inputs. An ensemble model may integrate predictions from the one or more machine learning models. Integrating predictions from different models may improve classification and anatomical screening.

In another example, generating the diagnostic prediction using the one or more machine learning models may include simultaneously generating the screening prediction of the cardiac anatomy and the classification of the cardiovascular condition, and wherein the one or more machine learning models is at least one of: a single multitask neural network architecture, a cascading output model, a hybrid model, or an ensemble model.

In yet another example, the cardiovascular condition may include at least one of: a hypertrophic cardiomyopathy, a dilated cardiomyopathy, a coronary artery disease, a left ventricular non-compaction cardiomyopathy, a restrictive cardiomyopathy, a cardiac amyloidosis, a hypertensive heart disease, a myocarditis, an arrhythmogenic right ventricular cardiomyopathy, a pulmonary arterial hypertension, or an Ebstein's Anomaly.

In another example in accordance with the disclosure, the method may include identifying a CMR-negative case and diagnosing a patient with a pulmonary arterial hypertension disease without a right heart catheterization of the patient.

In an additional example in accordance with this disclosure, generating a diagnostic prediction using the one or more machine learning models may also include conditioning a large language model on at least one of a vision module or an imaging feature.

Another example in accordance with this disclosure may include processing the sequence of radiographic images by dynamically adjusting the sequence of radiographic images based on an availability of a contrast-enhanced sequence or using at least one of the cine MRI, the T1 mapping, the T2 mapping, the perfusion imaging, the flow quantification, the dark blood imaging, the real-time imaging, the magnetic resonance spectroscopy, or the parametric mapping sequences when the contrast-enhanced sequence is unavailable. Selecting and using may be performed automatically. For example, when a contrast-enhanced sequence is unavailable (e.g. for a contrast-intolerant patient), a dynamic adjustment of the sequence of radiographic images may include automatically selecting and using a cine MRI sequence in accordance with this disclosure.

In another example in accordance with this disclosure, processing may include employing image synthesis techniques to generate or enhance missing modalities (e.g., synthesizing a contrast-enhanced image from cine MRI or other available sequences). Processing may further include optimizing the diagnostic prediction across varying image types and conditions using a strategy. A strategy may include image fusion, sequence combination, or a generative model.

In another example in accordance with this disclosure, the one or more machine learning models further may include at least one of: a convolutional neural network, a transformer-based model, a vision-language hybrid model that may process both visual and textual inputs, a hybrid model combining CNN and transformer architectures, a large language model, a recurrent neural network (RNN), a generative adversarial network (GAN), a graph neural network (GNN), a multi-modal model which may combine multiple input types, a self-supervised learning model, a semi-supervised learning framework, an attention-based model, a reinforcement learning model, or a model that integrates patient data with imaging data. Patient data may include clinical records and genetic data. Integrating patient data with imaging data may improve diagnostic prediction. A reinforcement learning model may adaptively learn and improve the model. A large language model or natural language processing method may be used to generate a report, which may include a textual explanation of findings, a justification in natural language, a diagnostic impression, a functional metric, a hemodynamic assessment, a morphological characteristic, a myocardial fibrosis marker, a motion analysis, or a treatment recommendation, or a summary. The report may be derived from information from the sequence of radiographic images of the heart and may include information from the patient's clinical history, electronic health records, external references, and clinical standards. The summary may include a piece of information derived from the radiographic image sequence.

Yet another example in accordance with this disclosure may include the quantitative assessment of the cardiac function parameter which may be at least one of: a left ventricular ejection fraction (LVEF), a right ventricular ejection fraction (RVEF), a wall thickness, a cardiac output, an end-diastolic volume (EDV), a systolic volume (SV), an end-diastolic volume index (EDVi), a stroke volume, a wall motion index, a myocardial strain, a myocardial perfusion, a tissue characterization, a right ventricular volume, a left ventricular volume, a cardiac workload, a myocardial workload, a ventricular mass, a left atrial volume, a right atrial volume, or a left ventricular outflow tract (LVOT) velocity. Myocardial strain may include radial, circumferential, or longitudinal strain. A cardiac function parameter may also be another dynamic or static parameter indicative of cardiac function.

In another example in accordance with this disclosure, the structured radiographic report may include at least one of: a diagnostic impression, a functional metric, a hemodynamic assessment, a morphological characteristic, a myocardial fibrosis marker, a motion analysis, a treatment recommendation, a risk stratification assessment, a disease progression evaluation, a comparison with a prior imaging study, or a summary. The summary may include key findings. The structured radiographic report may further include a visual overlay, a segmented anatomical region, a temporal change in cardiac function, and an AI-generated insight. The AI-generated insight may enhance interpretability and clinical decision-making. Additionally, the report May incorporate an automated suggestion for treatment, a therapeutic intervention, a follow-up examination, or a personalized management plan based on the diagnostic prediction. The structured radiographic may also support integration with electronic health record (EHR) systems, enabling seamless updates to patient records, automated flagging for clinical review, and compatibility with telemedicine platforms.

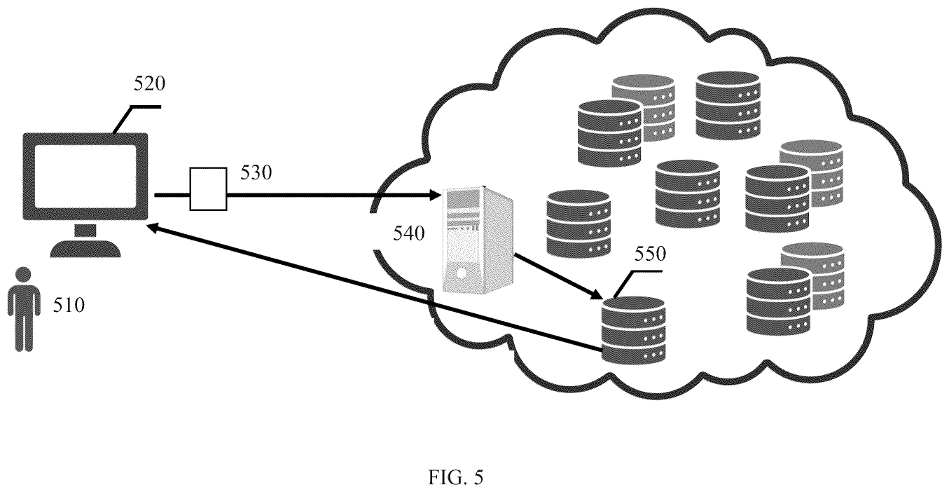

FIG. 5 is an illustration of a deployment of a screening and diagnostic system in accordance with one or more examples of the present disclosure. FIG. 5 may also be seen as a schematic diagram illustrating a cloud-based CMR interpretation system. In one example, the screening and diagnostic method may be flexibly deployed across various computing environments to support diverse clinical and commercial scenarios. One prominent deployment configuration may be via the cloud, where multiple computing nodes with allocated processing and storage resources operate collaboratively or independently to deliver diagnostic services. In a cloud-based setup, the service may be exposed via a software development kit (SDK) or an application programming interface (API), which may enable integration with third-party systems or front-end applications. These APIs may be invoked by user equipment such as desktop applications, web-based interfaces, mobile apps, or hospital imaging platforms. In FIG. 5, a user (e.g., patient, clinician, radiologist, or technician) 510 may interact with user equipment or an electronic interface 520 to initiate a cardiac MRI interpretation request. This request may include raw or preprocessed CMR sequences acquired from an MRI scanner. The interface 520 may transmit a CMR Image 530 to an API interface onto which one or more machine learning models 550 may be deployed on at least one of: a cloud-based application programming interface (API) or Management Node 540, an on-premise hospital server, a picture archiving and communication system (PACS), a Radiology Information System (RIS), an edge device for point-of-care diagnostics, a mobile platform, a distributed computing environment, or a federated learning framework. A cloud infrastructure may include a Management Node 540 that may be responsible for request routing and resource orchestration. The cloud infrastructure may also include a Computing Node 550: Each node may run one or more AI models (screening, diagnosis, cardiac function estimation, report generation, etc.). Upon receiving a request, the management node may select an available computing node based on workload or service matching.

The computing node may perform one or multiple functions as instructed by the user, including but not limited to,
Applying a deep learning model to output screening and diagnostic labels, cardiac function measurements, and segmentations.
Generating a structured radiographic report and/or natural language summary with key findings.
Providing a visual explanation and diagnostic rationale to support interpretability.
Returning an enriched result to the user interface for review, editing, or downstream clinical decisions.

The response from the cloud may be rendered on a platform, including a web dashboard, PACS viewer, or a mobile application, thus offering flexibility for both a real-time and offline interpretation workflow.

Beyond the cloud, the system can also be deployed in other configurations, including
1. On-Premise Hospital Servers:
   For institutions with regulatory or latency requirements, the AI engine may be integrated directly into local infrastructure.
   May enable real-time inference with secure data handling within institutional firewalls.
2. Embedded in PACS/RIS or Imaging Workstations:
   The AI functionality may be embedded within radiology imaging software to automatically launch upon image loading, providing real-time overlays, alerts, and recommendations.
3. Edge Devices (e.g., Compact Workstations in Low-Resource Settings):
   Lightweight versions of the AI model may be installed on portable diagnostic stations, useful in rural clinics or mobile screening units.

The deployment has potential applications and benefits, including:
A Clinical Workflow Automation, which may enable automated triage, report generation, and follow-up recommendations to reduce radiologist workload and report turnaround times.
A Global Health Impact, which may support a diagnosis in underserved or resource-constrained environments where expert radiologists are not available.
Assistance with a Second-Opinion which may provide consistent, evidence-based AI output to augment decision-making and reduce inter-reader variability.
Integration with EHR Systems, via API, which output may be linked directly to patient records to enhance longitudinal care planning.
A Population Screening Program, such as a cine-only model variant which may support scalable, contrast-free CMR screening initiatives for early disease detection.
A Remote Patient Access, where patients or users may upload their CMR images to the online platform, and the AI system may process them and provide CMR interpretation results, which may enable healthcare services without the need for hospital visits.

The deployment may enable a seamless integration with an electronic health record (EHR) system, a remote monitoring platform, a telemedicine service, or a multi-site diagnostic network, and may support both synchronous and asynchronous interpretation workflows. A CMR image 530 may be marked one or more of a diagnostic label, region segmentation, and a report that includes cardiac functions, region segmentations, key findings, and diagnostic rationales. After processing by the machine learning model on the Computing Node 550, an output may be transmitted to the electronic interface 520, an electronic health record, a cloud-based platform, or a mobile application.

The screening and diagnostic system may be configured to support at least one of the following use cases:

A Cloud-Based Platform which may be executed through distributed computing nodes accessible via application programming interfaces (APIs), software development kits (SDKs), or secure web portals, and may enable scalable remote access, telemedicine applications, or multi-institutional data sharing.

An edge device and on-premise server, which may be deployable in localized medical facilities, mobile clinics, or low-resource settings, enabling real-time, offline analysis without reliance on continuous cloud connectivity.

An integration with Clinical Decision Support Systems (CDSS), which may facilitate automated follow-up recommendations, triage prioritization, risk stratification, and clinical decision-making based on generated diagnostic rationale, cardiac function metrics, and screening predictions.

An Interactive Web-Based Visualization, which may allow users to dynamically explore visual maps, attention overlays, cardiac function metrics, diagnostic rationales, and textual justifications through secure, interactive dashboards.

A Patient-Facing Platform, which may provide patients with secure, direct access to their diagnostic results, cardiac function assessments, and interpretability features via online portals or mobile applications, enabling remote consultation, second opinions, and longitudinal health tracking.

A deployment configuration may support real-time updates, interactive exploration of diagnostic data, seamless integration with existing healthcare infrastructure, and optimized clinical workflow.

In an example, in accordance with this disclosure, the user equipment 520 sends a CMR image 530 request to the cloud, where the management node 540 routes the request to an appropriate computing node 550. The computing node 550 processes the image 530, generates diagnostic outputs and reports, and sends the results back to the user equipment 520 for display and further clinical use.

In another example, deployment options for portions of the systems and methods of this disclosure may include integration into Picture Archiving and Communication Systems (PACS), hospital servers, or cloud-based APIs 540. Such deployments may make the system suitable for high-volume hospitals as well as resource-constrained environments, such as smaller hospitals who may not have resources to implement a deployment of an example according to this disclosure on-site.

It is appreciated that the above described examples may be implemented by hardware, or software (program codes), or a combination of hardware and software. When implemented by software, instructions may be stored in computer-readable media. Software, when executed by the processor, may perform the disclosed methods. The computing units and other functional modules described in this disclosure may be implemented by hardware, software, or a combination thereof. Multiples of the above-described modules/units may be combined as one module/unit, and each of the above-described modules/units may be further divided into a plurality of sub-modules/sub-units.

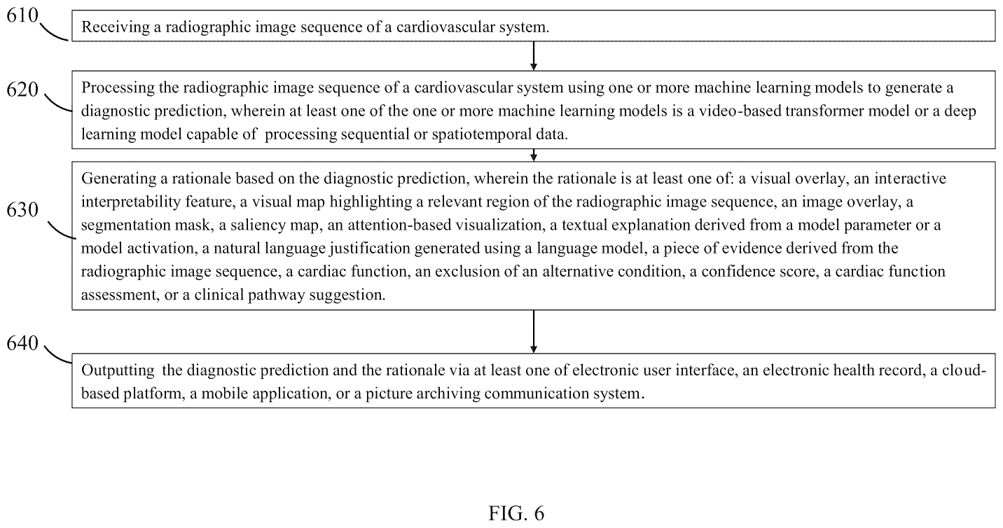

FIG. 6 is a flowchart illustrating a computer-implemented method for analysis of cardiac magnetic resonance imaging in accordance with an example of the disclosure.

Step 610 includes receiving a radiographic image sequence of a cardiovascular system.

Step 620 includes processing the radiographic image sequence of a cardiovascular system using one or more machine learning models to generate a diagnostic prediction, wherein at least one of the one or more machine learning models is a video-based transformer model or a deep learning model capable of processing sequential or spatiotemporal data.

Step 630 includes generating a rationale based on the diagnostic prediction, wherein the rationale is at least one of: a visual overlay, an interactive interpretability feature, a visual map highlighting a relevant region of the radiographic image sequence, an image overlay, a segmentation mask, a saliency map, an attention-based visualization, a textual explanation based on a model parameter or a model activation, a natural language justification generated using a language model, a piece of evidence derived from the radiographic image sequence, a cardiac function, an exclusion of an alternative condition, a confidence score, a cardiac function assessment, or a clinical pathway suggestion.

Step 640 includes outputting the diagnostic prediction and the rationale via at least one of an electronic interface, an electronic health record system, a cloud-based platform, a mobile application, or a picture archiving communication system.

Any number of additional steps, functions, processes, or variants thereof may be included in the method, including any disclosed relative to any other figure of this disclosure.

In another example, the method may include selecting a treatment based on the diagnostic prediction and the rationale.

In another example, processing the radiographic image sequence of a heart further May include extracting a region of interest from the radiographic image sequence. A region of interest (ROI) may encompass the heart, great vessels, or surrounding anatomical structures from the radiographic image sequence. Processing may further include performing segmentation of cardiac chambers, myocardium, and vascular structures, which may facilitate feature extraction and diagnostic analysis. Further processing may include applying noise reduction, motion correction, and image enhancement techniques, which may improve image quality and model interpretability.

An example of this disclosure may be expressed as a computerized system for automated interpretation of cardiac magnetic resonance imaging. The system may include a computerized device which may have a non-transitory memory, one or more processing apparatuses that May be in communication with the non-transitory memory, and a computer readable storage medium.

The system may further include a magnetic resonance imaging (MRI) machine which may be in communication with the computerized device. The MRI machine may be configured to acquire a sequence of radiographic images of a cardiovascular system. The sequence of radiographic images may be at least one of: a cine MRI, a late gadolinium enhancement sequence, a T1 mapping, a T2 mapping, a perfusion imaging, a flow quantification (e.g., 4D Flow MRI), a dark blood imaging, a real-time imaging, a spectroscopy (e.g., magnetic resonance spectroscopy), or a parametric mapping sequence. The sequence of radiographic images May include another cardiac MRI imaging modality or sequence that provides relevant structural or functional information of the cardiovascular system.

The system may also have one or more programs with program instructions that may be stored on the computer readable storage medium and may be executable by the one or more processing apparatus via the non-transitory memory. The instructions may include processing the sequence of radiographic images using one or more machine learning models. At least one of the one or more machine learning models may be a video-based transformer model, a convolutional neural network (CNN), a recurrent neural network (RNN), a deep neural network (DNN), or a graph-based neural network. The one or more machine learning models may be a video-based swin transformer or another model capable of processing sequential or spatiotemporal data.

The instructions may further include generating a diagnostic prediction using the one or more machine learning models. The diagnostic prediction may be at least one of a screening assessment of a cardiac anatomy, a diagnostic identification or suggestion of a cardiovascular condition, a quantitative evaluation of a cardiac function parameter, a structured radiographic report, a natural language summary of findings, or a diagnostic rationale.

The diagnostic rationale may be presented as a visual overlay, a textual explanation, or an interactive interpretability feature, or a combination thereof.

Further, the instructions may include outputting the diagnostic prediction to at least one of an electronic user interface, an electronic health record, a cloud-based platform, a mobile application, a picture archiving and communications system, or any other computing system capable of displaying or storing medical data.

In another example, the instructions may include selecting, recommending, or suggesting a treatment, intervention, follow-up examination, or follow-up actions based on the diagnostic prediction.

In another example in accordance with this disclosure, processing the sequence of radiographic images may further include extracting a region of interest from the sequence of radiographic images.

In yet another example in accordance with this disclosure, generating the diagnostic prediction using the one or more machine learning models may further include sequentially generating the screening prediction of the cardiac anatomy and a classification of the cardiovascular condition. The one or more machine learning models may be at least one of: a single multitask neural network architecture or a cascading output.

In another example in accordance with this disclosure, the diagnostic suggestion of a cardiovascular condition may be at least one of a hypertrophic cardiomyopathy, a dilated cardiomyopathy, a coronary artery disease, a left ventricular non-compaction cardiomyopathy, a restrictive cardiomyopathy, a cardiac amyloidosis, a hypertensive heart disease, a myocarditis, a arrhythmogenic right ventricular cardiomyopathy, a pulmonary arterial hypertension, or a congenital heart disease.

In another example of this disclosure, a computerized system for automated diagnosis of cardiovascular diseases may use a two-stage deep learning pipeline. The system may have a computerized device that may have a non-transitory memory, one or more processing apparatuses in communication with the non-transitory memory, and a computer readable storage medium. The system may further include a magnetic resonance imaging (MRI) machine in communication with the computerized device. The MRI machine may be configured to acquire a cine MRI sequence of radiographic images of a cardiovascular system without a contrast agent. Further, the system may include one or more programs that may have program instructions stored on the computer readable storage medium and executable by the one or more processing apparatus via the non-transitory memory. The instructions may include processing the cine MRI sequence of radiographic images using one or more machine learning models, wherein at least one of the one or more machine learning models may be a video-based swin transformer. Further, the instructions may include detecting a cardiac anomaly using the cine MRI sequence of radiographic images in a first stage. The instructions may also include generating a diagnostic classification using the cine MRI sequence of radiographic images and a late gadolinium enhancement MRI in a second stage.

In another example, the diagnostic classification of the system may be at least one of hypertrophic cardiomyopathy, dilated cardiomyopathy, coronary artery disease, left ventricular non-compaction cardiomyopathy, restrictive cardiomyopathy, cardiac amyloidosis, hypertensive heart disease, myocarditis, arrhythmogenic right ventricular cardiomyopathy, pulmonary arterial hypertension, a valvular heart disease (including aortic stenosis, mitral regurgitation, and tricuspid insufficiency), a pericardial disease (such as constrictive pericarditis and pericardial effusion), an ischemic heart disease, heart failure (systolic or diastolic), myocardial infarction, a vascular abnormality, another structural, functional, or ischemic abnormality, or a congenital heart disease, including Ebstein's Anomaly Tetralogy of Fallot, and atrial or ventricular septal defects as non-limiting examples.

In another example, the system and/or methods also may support multimodal data integration, which may combine imaging with demographic, clinical, or laboratory data. This combination may improve diagnostic performance.

In an example in accordance with this disclosure, a system may support various input cardiac MRI modalities-which may include short-axis and long-axis cine MRI, LGE, and T1, T2, and parametric mapping—and may be designed to be modality-adaptive. This adaptive design may allow for flexible configurations based on input availability. For example, in simplified acquisition settings, the present disclosure may support screening and/or diagnostics based on single cine views or selected representative slices, which may reduce scan time and improve throughput in addition to other benefits identified in this disclosure.

In another example, the system may generate a diagnostic rationale, which may enhance interpretability of the output and clinical trust. For each prediction, the system may generate a rationale that may include visual overlays, an image overlay or segmentation mask, a saliency map, an attention-based visualization, image-derived clinical evidence, and differential diagnoses outlining the exclusion of alternative conditions. A differential diagnosis may indicate an alternative condition considered and excluded. The rationale may include a textual explanation derived from a model parameter or activation. A natural language justification may be generated using a language model. Supporting evidence may be extracted from the radiographic image sequence. The rationale may further include a confidence score reflecting the reliability of the prediction, and which may be derived from model uncertainty estimation or statistical analysis. The rationale may also include a cardiac function assessment that may support the diagnosis, including but not limited to: Left ventricular ejection fraction (LVEF), Right ventricular ejection fraction (RVEF), Left ventricle and right ventricle volume indices, cardiac output, stroke volume, and wall motion abnormalities, strain analysis (radial, circumferential, longitudinal), myocardial perfusion and tissue characterization, time-series analysis of functional parameters over cardiac cycles. The rationale may also include a clinical pathway suggestion for follow-up examination, treatment planning, or preventive care, including a referral, additional imaging, or a therapeutic intervention.

Another example of the disclosure may be a system for analysis of cardiac magnetic resonance imaging. The system may have a computerized device that may have a non-transitory memory, one or more processing apparatuses in communication with the non-transitory memory, and a computer readable storage medium.

The system may further include a magnetic resonance imaging (MRI) machine in communication with the computerized device. The MRI machine may be configured to acquire a sequence of radiographic images of a heart.

Further, the system may have one or more programs including program instructions stored on the computer readable storage medium and executable by the one or more processing apparatus via the non-transitory memory. The instructions may include receiving the sequence of radiographic images of the heart. Further, the instructions may include processing the sequence of radiographic images of the heart using one or more machine learning models to generate a diagnostic prediction, wherein at least one of the one or more machine learning models is a video-based swin transformer. Additionally, the instructions may include generating, by the one or more machine learning models, a rationale based on the diagnostic prediction. The rationale may be at least one of a visual map highlighting a relevant region of the sequence of radiographic images of the heart, a saliency map, an image overlay, an explanation based on a model activation, a natural language justification generated using a language model, a piece of information supporting the diagnosis, or an exclusion of a condition, wherein the piece of information is derived from the sequence of radiographic images of the heart. The rationale May also include the exclusion of alternative conditions based on information which may include, for example, a prediction value or confidence that is higher or lower than a threshold value or lower than a prediction value of other cardiovascular diseases.

Further, the instructions may include providing outputting the diagnostic prediction and the rationale via at least one of an electronic user interface or an electronic health record system.

The disclosure may also incorporate a device which may include a magnetic resonance imaging (MRI) device. The MRI device may be configured to collect a cine MRI sequence of radiographic images of a heart of a patient without a contrast and to collect a late gadolinium enhancement (LGE) MRI of the heart of the patient. The device may also include at least one of an electronic user interface or an electronic health record. The device may also include an article comprising one or more machine readable storage media storing instructions, which may be operable to cause one or more machines to perform operations. The operations may include processing the cine MRI sequence of radiographic images using one or more machine learning models. Additionally, at least one of the one or more machine learning models may be a video-based swin transformer. Further, the instructions may include determining a cardiac anomaly using the cine MRI sequence of radiographic images in a first stage, wherein the cine MRI sequence of radiographic images may be at least one of: a short-axis view, a long-axis view, a four-chamber view, a three-chamber view, or a representative slice. A combination or representative slices may capture anatomical and functional characteristics of a subject's cardiovascular system, including a standard or a non-standard imaging plane.

The instructions may further include identifying a diagnostic classification using the cine MRI sequence of radiographic images and the LGE MRI in a second stage. Additionally, the one or more machine learning models may integrate a temporal information and a spatial information from at least one of the cine MRI sequence and the LGE MRI. Further, the diagnostic classification may be at least one of hypertrophic cardiomyopathy, dilated cardiomyopathy, coronary artery disease, left ventricular non-compaction cardiomyopathy, restrictive cardiomyopathy, cardiac amyloidosis, hypertensive heart disease, myocarditis, arrhythmogenic right ventricular cardiomyopathy, pulmonary arterial hypertension, or Ebstein's Anomaly.

Further, the instructions may include outputting the diagnostic classification to at least one of the electronic user interface or the electronic health record.

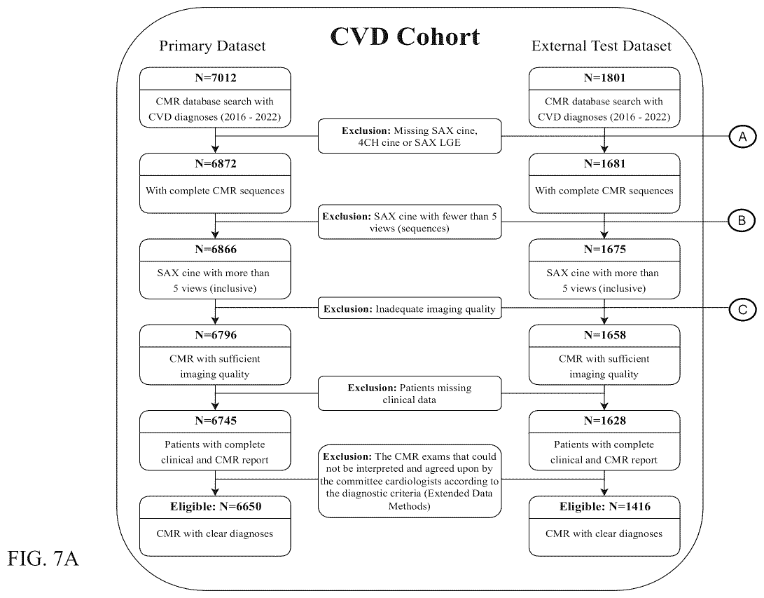

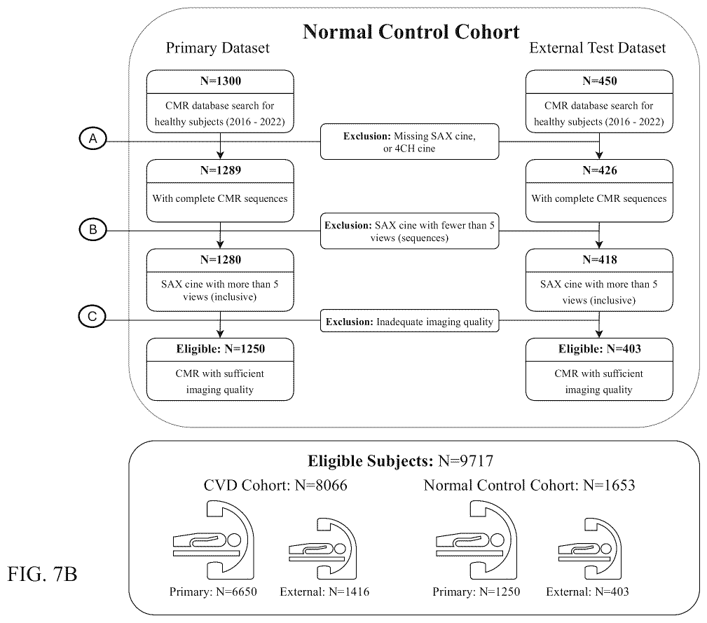

Referencing FIGS. 7A and 7B, this disclosure represents a nationwide, large, representative CMR dataset of 9,719 individuals (6,608 male, 3,111 female) from eight medical centers. The dataset was divided into the cardiovascular disease cohort (FIG. 7A) and the normal control cohort (FIG. 7B). The inclusion-exclusion cascade is summarized in FIGS. 7A and 7B. T1 and t@ mapping may be used to further curate data. The disease cohort included 8,066 patients with cardiovascular disease (mean±standard deviation age 47.2±15, 70% male, admitted between 2016 to 2022). Eleven types of CVDs were incorporated with the following distribution: hypertrophic cardiomyopathy-HCM (2715), dilated cardiomyopathy-DCM (1639), coronary artery disease-CAD (1241), left ventricular non-compaction cardiomyopathy —LVNC (321), restrictive cardiomyopathy-RCM (377), cardiac amyloidosis-CAM (358), hypertensive heart disease-HHD (509), myocarditis (153), arrhythmogenic right ventricular cardiomyopathy-ARVC (424), pulmonary arterial hypertension-PAH (200), and Ebstein's anomaly (129). The baseline CMR scan (pre-treatment) of each patient, with short-axis (SAX) cine, four-chamber (4CH) cine and SAX LGE all available, was collected to establish the disease cohort. In addition, the SAX cine and 4CH cine of 1653 normal subjects (age 38±15, 56% male, enrolled between 2016 to 2022) were collected to assemble the normal control cohort without CVDs, allowing us to develop and validate the non-invasive screening model. Table 1 contains the summary statistics and the demographics of the datasets.

TABLE 1
Characteristics of the primary and external test datasets.

| | | Primary Dataset | | | | | | External Test Dataset | | | | | | |
|---|---|---|---|---|---|---|---|---|---|---|---|---|---|---|
| | | No. of | Sex | | | | Age | No. of | Sex | | | | Age | Entire |
| | | Subjects | Male | | Female | | (Range) | Subjects | Male | | Female | | (Range) | Dataset |
| Total | | 7900 | 5380 | (68%) | 2520 | (32%) | 45 ± 16 | 1819 | 1228 | (68%) | 591 | (32%) | 47 ± 16 | 9719 |
| | | | | | | | (2-86) | | | | | | (1-88) | |
| Normal Control | | 1250 | 700 | (56%) | 550 | (44%) | 37 ± 14 | 403 | 230 | (57%) | 173 | (43%) | 41 ± 16 | 1653 |
| Cohort | | | | | | | (10-78) | | | | | | (6-79) | |
| Cardiovascular | | 6650 | 4680 | (70%) | 1970 | (30%) | 47 ± 15 | 1416 | 998 | (71%) | 418 | (29%) | 48 ± 16 | 8066 |
| Disease Cohort | | | | | | | (2-86) | | | | | | (1-88) | |
| 1 | HCM | 2327 | 1513 | (65%) | 814 | (35%) | 48 ± 14 | 388 | 260 | (67%) | 128 | (33%) | 51 ± 15 | 2715 |
| | | | | | | | (7-86) | | | | | | (9-86) | |
| 2 | DCM | 1435 | 1076 | (75%) | 359 | (25%) | 44 ± 15 | 204 | 140 | (69%) | 64 | (31%) | 50 ± 14 | 1639 |
| | | | | | | | (4-82) | | | | | | (8-76) | |
| 3 | CAD | 942 | 829 | (88%) | 113 | (12%) | 56 ± 11 | 299 | 269 | (90%) | 30 | (10%) | 56 ± 11 | 1241 |
| | | | | | | | (8-83) | | | | | | (24-88) | |
| 4 | LVNC | 291 | 192 | (66%) | 99 | (34%) | 39 ± 16 | 30 | 18 | (60%) | 12 | (40%) | 40 ± 14 | 321 |
| | | | | | | | (6-77) | | | | | | (11-65) | |
| 5 | RCM | 355 | 170 | (48%) | 185 | (52%) | 50 ± 20 | 22 | 13 | (59%) | 9 | (41%) | 38 ± 24 | 377 |
| | | | | | | | (7-85) | | | | | | (1-78) | |
| 6 | CAM | 220 | 156 | (71%) | 64 | (29%) | 56 ± 11 | 138 | 92 | (67%) | 46 | (33%) | 59 ± 9 | 358 |
| | | | | | | | (18-83) | | | | | | (29-82) | |
| 7 | HHD | 402 | 366 | (91%) | 36 | (9%) | 42 ± 13 | 107 | 88 | (82%) | 19 | (18%) | 45 ± 14 | 509 |
| | | | | | | | (12-75) | | | | | | (21-75) | |
| 8 | Myocarditis | 87 | 64 | (74%) | 23 | (26%) | 28 ± 11 | 66 | 48 | (73%) | 18 | (27%) | 26 ± 12 | 153 |
| | | | | | | | (14-69) | | | | | | (8-68) | |
| 9 | ARVC | 370 | 245 | (66%) | 125 | (34%) | 39 ± 14 | 54 | 37 | (68%) | 17 | (32%) | 40 ± 14 | 424 |
| | | | | | | | (9-74) | | | | | | (13-67) | |
| 10 | PAH | 134 | 36 | (27%) | 98 | (73%) | 32 ± 12 | 66 | 22 | (33%) | 44 | (67%) | 38 ± 17 | 200 |
| | | | | | | | (10-72) | | | | | | (10-72) | |
| 11 | Ebstein's | 87 | 33 | (38%) | 54 | (62%) | 34 ± 16 | 42 | 11 | (26%) | 31 | (74%) | 32 ± 14 | 129 |
| | Anomaly | | | | | | (2-63) | | | | | | (6-61) | |

HCM, hypertrophic cardiomyopathy; DCM, dilated cardiomyopathy; CAD, coronary artery disease; LVNC, left ventricular non-compaction; RCM, restrictive cardiomyopathy; CAM, cardiac amyloidosis; HHD, hypertensive heart disease; ARVC, arrhythmogenic right ventricular cardiomyopathy; PAH, pulmonary arterial hypertension.

FIG. 8 is an illustration of cardiac MRIs utilized in model development of the screening and diagnostic systems, methods, and device, in accordance with examples of the present disclosure. For the data acquisition, cardiac MRI was performed using three vendors with the following distribution: GE Healthcare (4569), Philips (3683), and Siemens (1467). Cine sequence was performed in short-axis orientation covering the whole left ventricle (SAX cine), as well as in long-axis covering the two-, three-, and four-chamber (4CH) view. All cine sequences were 25 frames (cardiac cycle). LGE images cover the left ventricle from the apex to the base (SAX LGE). Performance is reported as assessed from two major views of cine exam-SAX cine and 4CH cine, as well as SAX LGE (FIG. 8). In an example of this disclosure, acquiring the sequence of radiographic images of a heart or cardiovascular system may further include performing an MRI on an instrument from at least one of GE Healthcare, Philips, or Siemens.

The disclosure used the CMR data from a hospital as the primary dataset for model development and data pooled from all the other medical centers as external test sets. For both screening and diagnostics, threefold cross-validation was performed within the primary dataset to further validate performance. This involved a total of 7,900 subjects and 6,650 CVD patients from the primary dataset contributing to the training of the screening and diagnostic models, respectively. Each fold of cross-validation employed 5,267 patients for screening model training and 4,433 for diagnostic model training. Overall, the screening and diagnostic models were tested with 9,719 and 8,066 patients (internal and external), respectively, and included patients from eight medical centers and CMR acquired from three different MRI vendors.

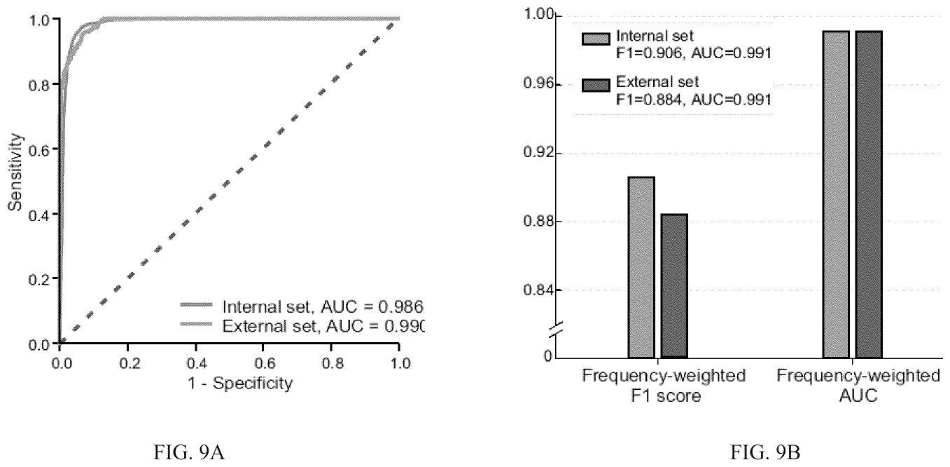

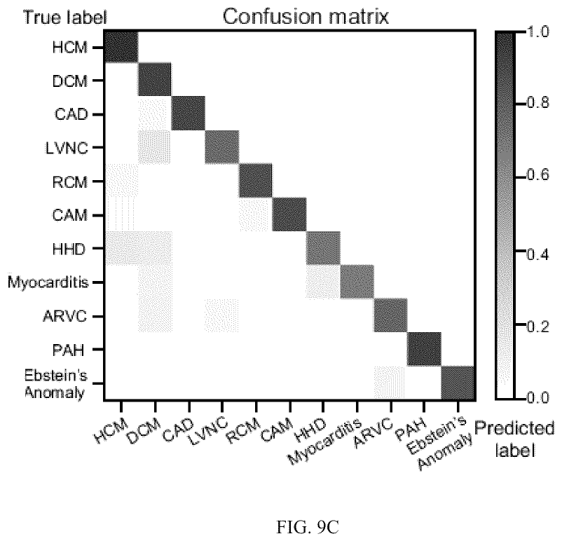

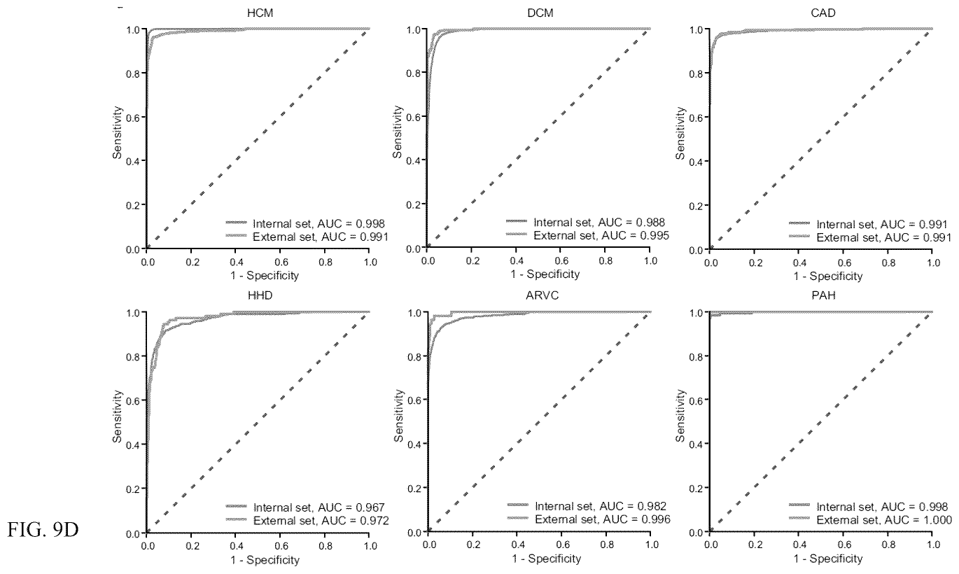

FIGS. 9A-9D and Table 4 show an evaluation of performance of the screening model. The screening model with cine MRI from two combined views (SAX cine and 4CH cine) achieved an AUC of 0.986 (95% Confidence Interval 0.984-0.988) and F1 score of 0.977 (95% CI 0.974-0.979) for screening on the threefold cross-validation upon the primary dataset (n=7900) (Table 3).

FIGS. 9A-9D are diagrammatical illustrations of performance curves characterizing the screening and diagnosis systems, methods and device, in accordance with examples of the present disclosure. FIG. 9A shows performance of the screening and diagnostic models in internal and external testing, with receiver-operating characteristic curves for the screening of cardiac anomalies for the primary internal test dataset (n=7,900) and external test dataset (n=1,819). The screening model is derived from four-chamber cine and short-axis cine.

FIG. 9B shows diagnostic performance for the internal test dataset (n=6,650) and external test dataset (n=1,416). The diagnostic model takes cine (4CH and SAX) and LGE as combined inputs.

FIG. 9C is a confusion matrix for the predictions of the AI diagnostic model versus the ground-truth over the entire cardiovascular disease cohort (n=8,066). The percentage of all possible predictions in each cardiovascular disease class is displayed on a color gradient scale.

FIG. 9D shows receiver-operating characteristic curves for the diagnosis of CVD classes for the internal set and external set. The CVD classes include HCM, hypertrophic cardiomyopathy; DCM, dilated cardiomyopathy; CAD, coronary artery disease; HHD, hypertensive heart disease; ARVC, arrhythmogenic right ventricular cardiomyopathy; PAH, pulmonary arterial hypertension. AUC stands for area under the curve. The sensitivity of 0.973 (0.968-0.978) was achieved by the model for anomaly detection with specificity at 90%. All sensitivity and specificity pairs were >90%. It is worth noting that the primary dataset contained a wide spectrum of CVDs (11 types; Table 1), demonstrating the robustness of the screening model with respect to disease type.

In the evaluation of each view of cine for screening, the model derived from four-chamber view received an AUC of 0.974 (95% CI 0.969-0.979); the model derived from short-axis view received an AUC of 0.971 (0.965-0.976). The combination of SAX and 4CH cine together provided the best performance in comparison to models derived from single view input (Table 3). Note that greater than 95% sensitivity was achieved by both single-view models for anomaly detection with specificity at 90% (Table 3). This demonstrates the potential of fast screening based on cine sequence from either SAX or 4CH view.

TABLE 3
Performance summary of the screening model for anomaly detection on the primary dataset (n =
7900; three-fold cross validation) and the external dataset (n = 1819) with different CMR input schemes.

| | SAX cine | | 4CH cine | | SAX + 4CH cine | |
|---|---|---|---|---|---|---|
| | Internal | External | Internal | External | Internal | External |
| AUROC | 0.971 | 0.953 | 0.974 | 0.980 | 0.986 | 0.990 |
| | (0.965-0.976) | (0.942-0.965) | (0.969-0.979) | (0.972-0.986) | (0.984-0.988) | (0.986-0.992) |
| PPV | 0.976 | 0.940 | 0.977 | 0.953 | 0.979 | 0.955 |
| | (0.972-0.980) | (0.928-0.952) | (0.974-0.981) | (0.941-0.964) | (0.976-0.983) | (0.944-0.966) |
| Specificity with | 0.975 | 0.916 | 0.975 | 0.940 | 0.986 | 0.970 |
| sensitivity at 90% | (0.966-0.983) | (0.873-0.949) | (0.966-0.983) | (0.914-0.964) | (0.978-0.993) | (0.950-0.990) |
| Sensitivity with | 0.956 | 0.909 | 0.967 | 0.941 | 0.973 | 0.959 |
| specificity at 90% | (0.\|951-0.963) | (0.886-0.934) | (0.962-0.973) | (0.910-0.962) | (0.968-0.978) | (0.936-0.974) |
| F1-score | 0.969 | 0.947 | 0.974 | 0.963 | 0.977 | 0.970 |
| | (0.966-0.972) | (0.939-0.955) | (0.971-0.977) | (0.955-0.970) | (0.974-0.979) | (0.964-0.977) |

AUROC, area under the receiver operating characteristic curve; PPV, positive predictive value (precision); CI, confidence interval; SAX, short axis; 4CH, four chamber.

The diagnostic model was also evaluated. This disclosure shows the use of the diagnostic model to classify eleven cardiovascular disease classes. Cine from both views (SAX and 4CH cine) and SAX LGE are combined inputs to the diagnostic model to ensure that any piece of complementary information present in CMR is effectively used to improve the diagnostic accuracy. Upon three-fold cross validation in the primary dataset (n=6650), the model achieved a class-weighted average AUC of 0.991 and F1 score of 0.906 (FIG. 9B; Table 4). The model achieved an AUC of greater than 0.96 for all classes; for all classes, all but three (LVNC, HHD and myocarditis) had F1 scores above 0.80. The model demonstrated high AUCs and F1 scores for the most prevalent CVDs including HCM (AUC: 0.998 [0.997-0.999]; F1: 0.975 [0.971-11 0.980]), DCM (0.988 [0.986-0.990]; 0.896 [0.884-0.907]), and CAD (0.991 [0.988-0.994]; 0.921 [0.908-0.935]). The PAH class also had a high AUC of 0.998 (0.995-1.000) and F1 score of 13 0.962 (0.937-0.984).

TABLE 4
Performance of the diagnostic models with different CMR input schemes
over three-fold cross validation of the primary dataset (n = 6650).

| | | AUROC (95% CI) | | | | | F1 score (95% CI) | | | | |
|---|---|---|---|---|---|---|---|---|---|---|---|
| | | | | SAX + | | | | | SAX + | | |
| | | SAX | 4CH | 4CH | | cine + | SAX | 4CH | 4CH | | cine + |
| Internal Testing | | cine | cine | cine | LGE | LGE | cine | cine | cine | LGE | LGE |
| 1 | HCM | 0.990 | 0.996 | 0.997 | 0.994 | 0.998 | 0.950 | 0.966 | 0.969 | 0.968 | 0.975 |
| | | (0.988- | (0.995- | (0.996- | (0.993- | (0.997- | (0.944- | (0.960- | (0.964- | (0.962- | (0.971- |
| | | 0.992) | 0.997) | 0.998) | 0.996) | 0.999) | 0.956) | 0.971) | 0.974) | 0.973) | 0.980) |
| 2 | DCM | 0.975 | 0.976 | 0.979 | 0.979 | 0.988 | 0.849 | 0.855 | 0.857 | 0.859 | 0.896 |
| | | (0.971- | (0.973- | (0.976- | (0.976- | (0.986- | (0.836- | (0.841- | (0.843- | (0.844- | (0.884- |
| | | 0.978) | 0.979) | 0.982) | 0.982) | 0.990) | 0.863) | 0.868) | 0.871) | 0.871) | 0.907) |
| 3 | CAD | 0.957 | 0.963 | 0.967 | 0.989 | 0.991 | 0.791 | 0.804 | 0.812 | 0.924 | 0.921 |
| | | (0.950- | (0.956- | (0.960- | (0.986- | (0.988- | (0.767- | (0.783- | (0.791- | (0.912- | (0.908- |
| | | 0.965) | 0.969) | 0.973) | 0.993) | 0.994) | 0.812) | 0.823) | 0.831) | 0.936) | 0.935) |
| 4 | LVNC | 0.943 | 0.961 | 0.970 | 0.958 | 0.978 | 0.657 | 0.760 | 0.784 | 0.637 | 0.778 |
| | | (0.928- | (0.949- | (0.960- | (0.942- | (0.970- | (0.610- | (0.719- | (0.744- | (0.584- | (0.739- |
| | | 0.957) | 0.972) | 0.979) | 0.975) | 0.986) | 0.703) | 0.799) | 0.821) | 0.681) | 0.816) |
| 5 | RCM | 0.959 | 0.984 | 0.992 | 0.978 | 0.994 | 0.732 | 0.865 | 0.870 | 0.769 | 0.873 |
| | | (0.945- | (0.975- | (0.987- | (0.967- | (0.991- | (0.696- | (0.836- | (0.842- | (0.733- | (0.847- |
| | | 0.970) | 0.991) | 0.995) | 0.989) | 0.997) | 0.769) | 0.893) | 0.896) | 0.805) | 0.900) |
| 6 | CAM | 0.980 | 0.981 | 0.986 | 0.990 | 0.994 | 0.787 | 0.868 | 0.869 | 0.884 | 0.918 |
| | | (0.970- | (0.969- | (0.976- | (0.980- | (0.988- | (0.738- | (0.833- | (0.836- | (0.850- | (0.888- |
| | | 0.988) | 0.990) | 0.994) | 0.999) | 0.998) | 0.829) | 0.903) | 0.900) | 0.914) | 0.943) |
| 7 | HHD | 0.929 | 0.947 | 0.955 | 0.955 | 0.967 | 0.662 | 0.672 | 0.676 | 0.696 | 0.723 |
| | | (0.915- | (0.935- | (0.944- | (0.940- | (0.958- | (0.621- | (0.634- | (0.641- | (0.660- | (0.684- |
| | | 0.942) | 0.958) | 0.965) | 0.969) | 0.976) | 0.698) | 0.707) | 0.714) | 0.732) | 0.757) |
| 8 | Myocar- | 0.940 | 0.963 | 0.970 | 0.964 | 0.987 | 0.480 | 0.576 | 0.615 | 0.590 | 0.724 |
| | ditis | (0.918- | (0.941- | (0.951- | (0.937- | (0.978- | (0.375- | (0.488- | (0.526- | (0.503- | (0.638- |
| | | 0.960) | 0.980) | 0.984) | 0.992) | 0.995) | 0.578) | 0.651) | 0.697) | 0.674) | 0.795) |
| 9 | ARVC | 0.965 | 0.969 | 0.976 | 0.968 | 0.982 | 0.721 | 0.775 | 0.780 | 0.757 | 0.816 |
| | | (0.956- | (0.960- | (0.967- | (0.959- | (0.975- | (0.681- | (0.740- | (0.746- | (0.717- | (0.787- |
| | | 0.973) | 0.978) | 0.983) | 0.976) | 0.988) | 0.758) | 0.809) | 0.813) | 0.794) | 0.846) |
| 10 | PAH | 0.997 | 0.995 | 0.998 | 0.994 | 0.998 | 0.939 | 0.893 | 0.923 | 0.951 | 0.962 |
| | | (0.992- | (0.990- | (0.997- | (0.986- | (0.995- | (0.907- | (0.853- | (0.888- | (0.922- | (0.937- |
| | | 1.000) | 0.998) | 1.000) | 1.003) | 1.000) | 0.968) | 0.932) | 0.954) | 0.977) | 0.984) |
| 11 | Ebstein's | 0.976 | 0.985 | 0.987 | 0.992 | 0.997 | 0.833 | 0.824 | 0.852 | 0.830 | 0.892 |
| | Anomaly | (0.954- | (0.969- | (0.968- | (0.979- | (0.994- | (0.766- | (0.758- | (0.789- | (0.761- | (0.832- |
| | | 0.993) | 0.996) | 0.999) | 1.005) | 1.000) | 0.887) | 0.880) | 0.907) | 0.890) | 0.935) |

| | | | | | | | | | | |
|---|---|---|---|---|---|---|---|---|---|---|
| Class frequency- | 0.972 | 0.979 | 0.983 | 0.983 | 0.991 | 0.838 | 0.865 | 0.871 | 0.875 | 0.906 |
| weighted average | | | | | | | | | | |

AUROC, area under the receiver operating characteristic curve; CMR, cardiac magnetic resonance imaging; SAX, short-axis; 4CH, four chamber; LGE, late gadolinium enhancement.
The bold font emphasizes the optimal performance metric among various input schemes.

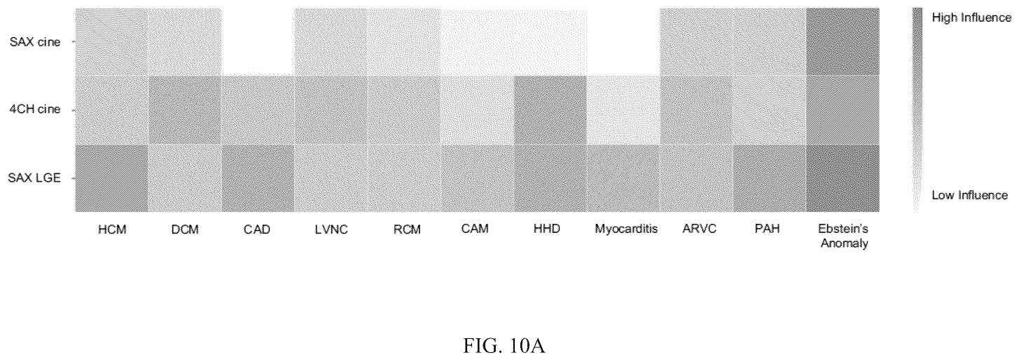

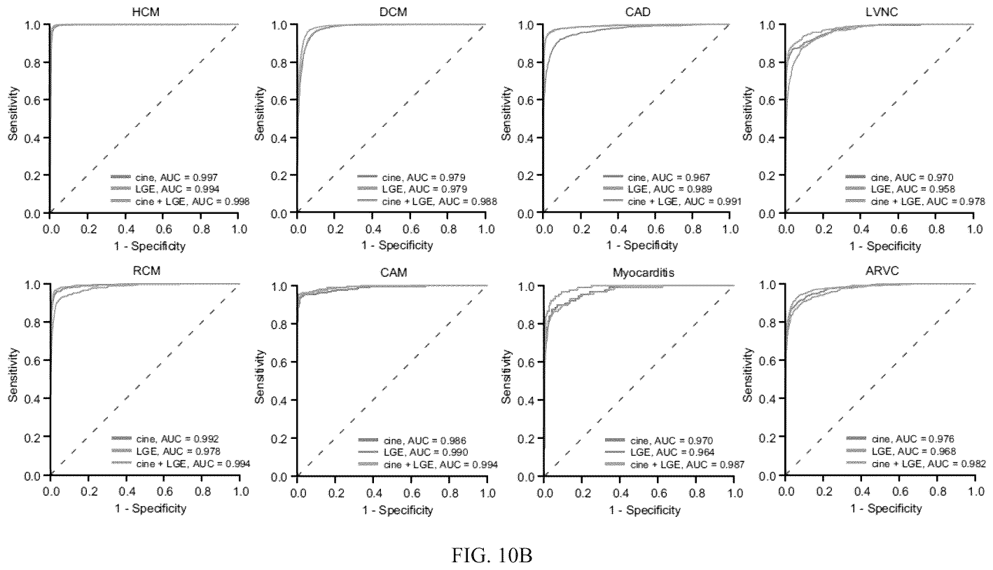

FIGS. 10A-10B are diagrammatical illustrations depicting additional characterizations of the screening and diagnostic methods, systems, and device in accordance with examples of the present disclosure. The disclosure examines the five input schemes: (1) SAX cine, (2) 4CH cine, (3) SAX and 4CH cine, (4) SAX LGE, and (5) the combination of SAX cine, 4CH cine, and SAX LGE. The all-input scenario (number (5) achieved the highest AUC and F1 across all eleven disease classes (FIG. 10A; Table 4). FIG. 10A represents influences of individual CMR modalities. In FIG. 10A, Shapley values for each of short-axis cine, four-chamber cine, and short-axis LGE, derived from the diagnostic model (cine and LGE as combined inputs), are presented for the prediction of each of the eleven cardiovascular disease classes. Shapley values are displayed on a greyscale gradient scale, with darker greys indicating the CMR modality with the greatest influence for each CVD classification. The CMR modalities, exhibiting characteristic features for the diagnosis of the cardiovascular disease class, demonstrate an impact on its model prediction: SAX LGE for the diagnosis of CAD (distinct feature: the endomyocardial or transmural LGE matching the area of coronary artery dominance); SAX LGE for HCM (hypertrophy and right ventricular insertion point LGE); SAX LGE for myocarditis (epicardial LGE); 4CH cine for LVNC (left ventricular noncompaction in the apex); 4CH cine for RCM (bi-atrial enlargement on the four-chamber view).

FIG. 10B shows receiver operating characteristic curves from the diagnostic models based on cine (darkest), LGE (mid), and cine+LGE as combined inputs (lightest). Combining cine and LGE yielded the optimal diagnostic performances for all CVD classes. The performance was based on the internal test set. In these figures, CVD is cardiovascular disease; LGE is late gadolinium enhancement; CMR is cardiovascular magnetic resonance imaging; SAX is short-axis; 4CH is four-chamber; and AUC is area under the curve. Receiver operating characteristic curves (ROCs) were plotted for the eleven disease classes. FIG. 10B presents the ROCs of three input schemes (cine, LGE, cine+LGE). Notably, the combination of cine and LGE MRIs significantly outperforms models derived from any single modality, with 1.9% points improvement in averaged AUC metric and 6.8% points improvement in averaged F1 metric (compared to SAX cine). All sensitivity and specificity pairs were >90% (Table 5). The positive predictive value (PPV) and negative predictive value (NPV) scores are provided in Table 7. Table 7 shows PPV and NPV of the diagnostic model derived from cine and LGE as combined inputs in the primary dataset (n=6650).

TABLE 5
Sensitivity and specificity analysis of the diagnostic
model derived from cine and LGE as combined inputs.

| | | Sensitivity (Specificity = 0.9) | | Specificity (Sensitivity = 0.9) | |
|---|---|---|---|---|---|
| | | Internal | External | Internal | External |
| 1 | HCM | 1.000 (0.999-1.000) | 0.979 (0.965-0.993) | 0.996 (0.994-0.997) | 0.986 (0.973-0.994) |
| 2 | DCM | 0.982 (0.975-0.989) | 0.995 (0.984-1.000) | 0.967 (0.961-0.973) | 0.988 (0.973-0.999) |
| 3 | CAD | 0.979 (0.968-0.988) | 0.987 (0.970-0.997) | 0.991 (0.987-0.995) | 0.987 (0.977-0.996) |
| 4 | LVNC | 0.938 (0.908-0.964) | 1.000 (1.000-1.000) | 0.948 (0.906-0.980) | 1.000 (0.993-1.000) |
| 5 | RCM | 0.986 (0.972-0.997) | 1.000 (1.000-1.000) | 0.991 (0.986-0.994) | 0.994 (0.927-0.999) |
| 6 | CAM | 0.973 (0.950-0.992) | 0.978 (0.951-1.000) | 0.998 (0.996-0.999) | 0.986 (0.968-1.000) |
| 7 | HHD | 0.920 (0.892-0.945) | 0.953 (0.906-0.991) | 0.923 (0.883-0.949) | 0.934 (0.908-0.955) |
| 8 | Myocarditis | 0.966 (0.923-1.000) | 0.955 (0.885-1.000) | 0.971 (0.922-0.993) | 0.955 (0.885-1.000) |
| 9 | ARVC | 0.949 (0.924-0.971) | 0.981 (0.944-1.000) | 0.963 (0.940-0.977) | 0.992 (0.981-1.000) |
| 10 | PAH | 0.993 (0.976-1.000) | 1.000 (1.000-1.000) | 1.000 (0.999-1.000) | 1.000 (1.000-1.000) |
| 11 | Ebstein's Anomaly | 0.989 (0.962-1.000) | 1.000 (1.000-1.000) | 0.999 (0.995-1.000) | 1.000 (1.000-1.000) |

*95% confidence interval in the brackets. HCM, hypertrophic cardiomyopathy; DCM, dilated cardiomyopathy; CAD, coronary artery disease; LVNC, left ventricular non-compaction; RCM, restrictive cardiomyopathy; CAM, cardiac amyloidosis; HHD, hypertensive heart disease; ARVC, arrhythmogenic right ventricular cardiomyopathy; PAH, pulmonary arterial hypertension.

TABLE 7
PPV and NPV of the diagnostic model derived from cine and
LGE as combined inputs in the primary dataset (n = 6650).

| | | PPV | | NPV | |
|---|---|---|---|---|---|
| | | Internal | External | Internal | External |
| 1 | HCM | 0.956 | 0.932 | 0.997 | 0.983 |
| | | (0.947-0.963) | (0.907-0.955) | (0.996-0.999) | (0.975-0.991) |
| 2 | DCM | 0.875 | 0.754 | 0.977 | 0.998 |
| | | (0.858-0.892) | (0.702-0.803) | (0.973-0.981) | (0.996-1.000) |
| 3 | CAD | 0.940 | 0.952 | 0.984 | 0.966 |
| | | (0.924-0.954) | (0.928-0.977) | (0.981-0.987) | (0.954-0.976) |
| 4 | LVNC | 0.805 | 1.000 | 0.989 | 0.994 |
| | | (0.757-0.848) | (1.000-1.000) | (0.986-0.991) | (0.989-0.998) |
| 5 | RCM | 0.877 | 0.600 | 0.993 | 0.999 |
| | | (0.843-0.912) | (0.433-0.767) | (0.990-0.995) | (0.998-1.000) |
| 6 | CAM | 0.951 | 0.983 | 0.996 | 0.985 |
| | | (0.921-0.979) | (0.955-1.000) | (0.995-0.998) | (0.978-0.991) |
| 7 | HHD | 0.746 | 0.735 | 0.981 | 0.976 |
| | | (0.704-0.789) | (0.644-0.823) | (0.977-0.984) | (0.967-0.983) |
| 8 | Myocarditis | 0.776 | 0.810 | 0.996 | 0.977 |
| | | (0.676-0.862) | (0.686-0.921) | (0.994-0.997) | (0.969-0.984) |
| 9 | ARVC | 0.864 | 0.904 | 0.987 | 0.995 |
| | | (0.825-0.899) | (0.816-0.977) | (0.984-0.989) | (0.991-0.999) |
| 10 | PAH | 0.992 | 1.000 | 0.999 | 0.997 |
| | | (0.974-1.000) | (1.000-1.000) | (0.998-0.999) | (0.994-0.999) |
| 11 | Ebstein's | 0.937 | 0.977 | 0.998 | 1.000 |
| | Anomaly | (0.875-0.986) | (0.918-1.000) | (0.997-0.999) | (1.000-1.000) |

*95% confidence interval in the brackets.
PPV: positive predictive value; NPV: negative predictive value.

Referring back to FIGS. 9A-9D, the model may be generalized to an external test set. To assess whether the models in this disclosure could be transferred to different institutions with varying data collection protocols, the screening and diagnostic models were validated on external test sets collected from seven medical centers (n=1819; 403 normal subjects and 1416 patients of CVDs). The screening model for anomaly detection attained an AUC of 0.990 (95% CI 0.986-0.992), F1 score of 0.970 (0.964-0.977), sensitivity of 0.959 (0.936-0.974) with specificity at 90%, and specificity of 0.970 (0.950-0.990) with sensitivity at 90% (FIG. 9A; Table 3). The diagnostic model (with all-input scenario) for CVD classification achieved a class-weighted AUC of 0.991 and F1 score of 0.884 (FIG. 9B; Table 6). This indicates that the AI model can generalize across diverse data sources, including medical centers uninvolved during model development.

TABLE 6
Performance of the diagnostic models with different CMR
input schemes over the external test dataset (n = 1416).

| | | AUROC (95% CI) | | | | | F1 score (95% CI) | | | | |
|---|---|---|---|---|---|---|---|---|---|---|---|
| | | | | SAX + | | | | | SAX + | | |
| External | | SAX | 4CH | 4CH | | cine + | SAX | 4CH | 4CH | | cine + |
| Testing | | cine | cine | cine | LGE | LGE | cine | cine | cine | LGE | LGE |
| 1 | HCM | 0.972 | 0.976 | 0.979 | 0.981 | 0.991 | 0.865 | 0.880 | 0.894 | 0.898 | 0.944 |
| | | (0.961- | (0.966- | (0.971- | (0.974- | (0.986- | (0.840- | (0.855- | (0.870- | (0.872- | (0.928- |
| | | 0.980) | 0.984) | 0.987) | 0.988) | 0.995) | 0.889) | 0.902) | 0.914) | 0.919) | 0.960) |
| 2 | DCM | 0.985 | 0.978 | 0.987 | 0.968 | 0.995 | 0.860 | 0.834 | 0.878 | 0.704 | 0.856 |
| | | (0.979- | (0.969- | (0.981- | (0.958- | (0.992- | (0.821- | (0.795- | (0.844- | (0.658- | (0.821- |
| | | 0.991) | 0.985) | 0.992) | 0.977) | 0.998) | 0.892) | 0.872) | 0.910) | 0.747) | 0.887) |
| 3 | CAD | 0.952 | 0.960 | 0.967 | 0.973 | 0.991 | 0.783 | 0.814 | 0.837 | 0.832 | 0.909 |
| | | (0.940- | (0.949- | (0.955- | (0.962- | (0.984- | (0.743- | (0.775- | (0.804- | (0.794- | (0.882- |
| | | 0.963) | 0.970) | 0.977) | 0.983) | 0.996) | 0.819) | 0.848) | 0.868) | 0.865) | 0.932) |
| 4 | LVNC | 0.962 | 0.994 | 0.997 | 0.962 | 1.000 | 0.638 | 0.691 | 0.814 | 0.512 | 0.824 |
| | | (0.906- | (0.987- | (0.995- | (0.923- | (0.999- | (0.491- | (0.533- | (0.690- | (0.293- | (0.683- |
| | | 0.994) | 0.998) | 0.999) | 0.990) | 1.000) | 0.772) | 0.821) | 0.913) | 0.667) | 0.931) |
| 5 | RCM | 0.951 | 0.997 | 0.997 | 0.914 | 0.995 | 0.433 | 0.667 | 0.688 | 0.333 | 0.737 |
| | | (0.903- | (0.994- | (0.995- | (0.840- | (0.988- | (0.250- | (0.519- | (0.548- | (0.121- | (0.583- |
| | | 0.987) | 0.999) | 0.999) | 0.967) | 0.999) | 0.586) | 0.789) | 0.813) | 0.536) | 0.852) |
| 6 | CAM | 0.951 | 0.973 | 0.977 | 0.977 | 0.992 | 0.782 | 0.852 | 0.859 | 0.827 | 0.915 |
| | | (0.927- | (0.957- | (0.964- | (0.964- | (0.986- | (0.727- | (0.803- | (0.810- | (0.773- | (0.877- |
| | | 0.973) | 0.986) | 0.989) | 0.989) | 0.997) | 0.839) | 0.897) | 0.904) | 0.872) | 0.949) |
| 7 | HHD | 0.927 | 0.926 | 0.937 | 0.917 | 0.972 | 0.687 | 0.690 | 0.694 | 0.654 | 0.718 |
| | | (0.898- | (0.894- | (0.909- | (0.878- | (0.959- | (0.608- | (0.616- | (0.624- | (0.571- | (0.644- |
| | | 0.953) | 0.955) | 0.963) | 0.951) | 0.983) | 0.759) | 0.759) | 0.761) | 0.725) | 0.789) |
| 8 | Myocar- | 0.917 | 0.913 | 0.943 | 0.951 | 0.972 | 0.438 | 0.391 | 0.458 | 0.605 | 0.630 |
| | ditis | (0.876- | (0.871- | (0.909- | (0.921- | (0.950- | (0.316- | (0.259- | (0.327- | (0.492- | (0.514- |
| | | 0.950) | 0.948) | 0.971) | 0.974) | 0.989) | 0.547) | 0.519) | 0.574) | 0.697) | 0.735) |
| 9 | ARVC | 0.985 | 0.973 | 0.982 | 0.965 | 0.996 | 0.723 | 0.706 | 0.748 | 0.729 | 0.887 |
| | | (0.972- | (0.951- | (0.951- | (0.940- | (0.992- | (0.625- | (0.607- | (0.649- | (0.621- | (0.813- |
| | | 0.994) | 0.990) | 0.997) | 0.984) | 0.999) | 0.800) | 0.793) | 0.830) | 0.818) | 0.948) |
| 10 | PAH | 0.999 | 0.969 | 0.999 | 0.993 | 1.000 | 0.930 | 0.814 | 0.900 | 0.880 | 0.969 |
| | | (0.998- | (0.941- | (0.999- | (0.985- | (1.000- | (0.880- | (0.731- | (0.833- | (0.811- | (0.936- |
| | | 1.000) | 0.991) | 1.000) | 0.999) | 1.000) | 0.971) | 0.887) | 0.954) | 0.936) | 0.993) |
| 11 | Ebstein's | 0.999 | 0.999 | 1.000 | 0.999 | 1.000 | 0.941 | 0.848 | 0.921 | 0.889 | 0.988 |
| | Anomaly | (0.998- | (0.998- | (1.000- | (0.997- | (1.000- | (0.886- | (0.764- | (0.853- | (0.817- | (0.961- |
| | | 1.000) | 1.000) | 1.000) | 1.000) | 1.000) | 0.987) | 0.918) | 0.974) | 0.952) | 1.000) |

| | | | | | | | | | | |
|---|---|---|---|---|---|---|---|---|---|---|
| Class frequency- | 0.964 | 0.967 | 0.975 | 0.970 | 0.991 | 0.794 | 0.802 | 0.831 | 0.792 | 0.884 |
| weighted average | | | | | | | | | | |

AUROC, area under the receiver operating characteristic curve; CMR, cardiac magnetic resonance imaging; SAX, short-axis; 4CH, four chamber; LGE, late gadolinium enhancement.
The bold font emphasizes the optimal performance metric among various input schemes.

In addition, this disclosure shows the generalizability of models derived from a single imaging modality. The diagnostic models based on cine (SAX and 4CH views) film and LGE achieved cross-institution F1 scores of 0.831 and 0.792, respectively (Table 6). For the screening task, the cross-institution performance was 0.953 (0.942-0.965) of AUC by model derived from SAX cine and 0.980 (0.972-0.986) by model of 4CH cine (Table 3). The findings were consistent with that of the primary dataset: the combination of SAX and 4CH cine provides the best performance for detecting cardiac anomalies; integrating cine and LGE yields the optimal diagnostic performance.

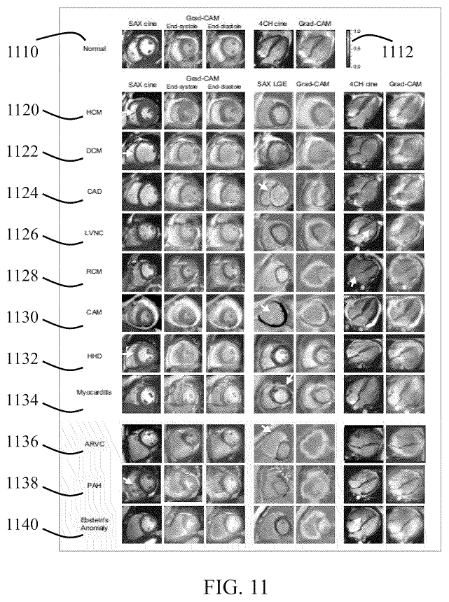

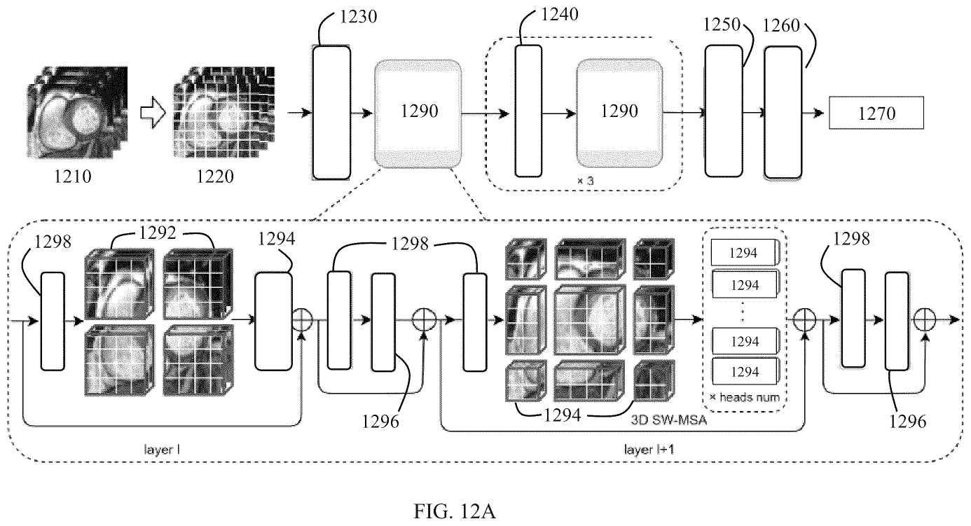

FIG. 11 is a series of images showing characterizations of the screening and diagnostic methods, systems, and device in accordance with examples of the present disclosure. The model may lead to interpretable images. The guided Grad-CAM approach was leveraged to display an informative set of features and distinct patterns used by the model for classification. Specifically, the Grad-CAM was extracted for representative subjects from eleven cardiovascular disease categories. FIG. 11 shows visual maps of the AI model activations that contributed to a prediction of cardiovascular disease. FIG. 11 represents saliency maps of CMR scans from representative patients of eleven CVD classes (1120-1140) and the normal control (1110). The saliency map (heatmap) was generated using the guided Grad-CAM approach and reveals the region that contributes the most to the AI model's decision. The scale bar 1112 ranges from zero to one, with one indicating the highest influence provided by the normalized Grad-CAM value, and zero indicating the lowest influence. The white arrows in each row of images point to the characteristic features of each CVD class, which are consistently encompassed by the saliency maps of the diagnostic model: left ventricular hypertrophy-HCM 1120 (arrow in SAX cine column); enlargement of the left ventricle and thinning of the left ventricular wall-DCM 1122 (arrow in SAW cine column); endocardial LGE in the ventricular septum and adjacent anterior of the left ventricular wall-CAD 1124 (arrow in SAX LGE column); left ventricular noncompaction in the apex-LVNC 1126 (arrow in 4CH cine column); biatrial enlargement—RCM 1128 (arrow in SAX LGE column); diffuse dust-like LGE of the left ventricular myocardium-CAM 1130 (arrow in SAX LGE column); symmetric left ventricular hypertrophy—HHD 1132 (arrow in SAX cine column); subepicardial LGE of the left ventricular free wall—Myocarditis 1134 (arrow in SAX LGE column); right ventricular enlargement with fibrosis—ARVC 1136 (arrow in SAX LGE column); enlargement of the right ventricle and thickening of the right ventricular wall-PAH 1138 (arrow in SAX cine column); apical displacement of the septal valve leaflet of the tricuspid valve-Ebstein's anomaly 1140 (arrow in 4CH cine column).

Grad-CAM, gradient-weighted class activation mapping. The CVD classes from 1120 through 1134 are primarily left ventricle dysfunctions and the classes from 1136 through 1140 are primarily right ventricle dysfunctions. The left ventricle area shows higher saliency at the detection of HCM 1120, DCM 1122, CAD 1124, LVNC 1126, RCM 1128, CAM 1130, HHD 1132 and myocarditis 1134. The right ventricle was highlighted as salient for the detection of ARVC 1136, PAH 1138, and Ebstein's anomaly 1140. This is consistent with the clinical diagnostic criteria: ARVC, PAH and Ebstein's anomaly are all primarily right ventricle involvement whereas the abnormality for the rest of the classes is mainly present on left ventricle. In addition, the LGE signal in CAD 1124, CAM 1130, myocarditis 1134, and ARVC 1136 (myocardium in SAX LGE, white arrows), which represents myocardial fibrosis or amyloid, was correctly captured by the saliency maps. Furthermore, the model accurately identified the left ventricular non-compaction in the apex and septal leaflet displacement as distinctive features in detecting LVNC 1126 and Ebstein's anomaly 1140 (4CH cine, white arrows), respectively, which is consistent with the underlying pathophysiology of these conditions.

Table 2 is a table comparing performance of physicians with the screening and diagnostic system and methods, in accordance with examples of the present disclosure. The performance of the AI model 270 with physicians of varying experience in CMR interpretation may be compared. The model performance may be compared with human annotations. To compare the performance of the AI model with that of board-certified physicians, a conventional test dataset was formed with 500 patients covering 11 types of CVDs. Each patient was independently evaluated for CVD class by physicians with three levels of experience in CMR reading (3-5 years, 5-10 years, and more than 10 years), along with the AI diagnostic model for comparison (Table 2). The AI model achieved comparable performance with physicians with more than years of experience in CMR reading (F1 score of 0.931 vs. 0.927) with faster speed of interpretation (1.94 minutes versus 418 minutes for interpreting 500 subjects). In addition, the model exceeded the performance of the most experienced group of physicians (more than 10 years) for the PAH class by successfully identifying CMR-negative patients (F1 score of 0.983 vs. 0.931). This demonstrates the potential of AI to identify MRI features not readily detectable by humans. The model performance matched or exceeded the most experienced physicians, but interpreted results for 500 patients in less than two minutes instead of almost seven hours. The improved identification of CMR-negative patients indicates the model is doing work not readily achievable by the human mind, even of the most highly skilled minds.

TABLE 2
Diagnostic performance of the AI model compared with physicians
with varying experience (range: 3 to >10 years) in CMR reading.

| | | No. of | F1 score | | | |
|---|---|---|---|---|---|---|
| | | Subjects | AI | Physician | Physician | Physician |
| | | (n = 500) | model | (3-5 years) | (5-10 years) | (>10 years) |
| 1 | HCM | 100 | 0.971 | 0.957 | 0.938 | 0.962 |
| 2 | DCM | 100 | 0.914 | 0.853 | 0.911 | 0.940 |
| 3 | CAD | 80 | 0.962 | 0.916 | 0.949 | 0.969 |
| 4 | LVNC | 30 | 0.877 | 0.667 | 0.778 | 0.885 |
| 5 | RCM | 30 | 0.933 | 0.578 | 0.760 | 0.800 |
| 6 | CAM | 30 | 0.947 | 0.667 | 0.931 | 0.931 |
| 7 | HHD | 30 | 0.833 | 0.615 | 0.667 | 0.896 |
| 8 | Myocarditis | 20 | 0.857 | 0.553 | 0.600 | 0.683 |
| 9 | ARVC | 30 | 0.897 | 0.451 | 0.814 | 0.983 |
| 10 | PAH | 30 | 0.983 | 0.061 | 0.929 | 0.931 |
| 11 | Ebstein's | 20 | 0.950 | 0.519 | 0.842 | 0.974 |
| | Anomaly | | | | | |

| | | | | |
|---|---|---|---|---|
| Frequency-weighted F1 | 0.931 | 0.734 | 0.872 | 0.927 |
| Accuracy | 0.932 | 0.746 | 0.868 | 0.928 |
| Time cost (in total) | 1.94 minutes | 576 minutes | 329 minutes | 418 minutes |

*Testing for the AI model was performed on 4 GeForce RTX 3090 GPUs.
The physicians are categorized according to their number of years of experience in CMR interpretation.
The bold font emphasizes the superior performance metric among subgroups, including the AI model and physicians with varying levels of experience.

FIGS. 12A and 12B show a schematic overview of a portion of a model used in the screening and diagnostic systems, methods and device, in accordance with examples of the present disclosure. The video-based swin transformer model and the conventional CNN-LSTM (Long short-term memory) approach were compared for modeling CMR sequences. FIGS. 12A and 12B illustrate the schematic overview of the two video-based deep learning algorithms, VST in FIG. 12A and CNN in FIG. 12B, in short-axis cine film interpretation. The SAX cine-derived VST model significantly outperformed CNN-LSTM with 3.5% points improvement in AUC and 4.6% points improvement in F1 score, tested upon the primary dataset. This finding demonstrates the superiority of video-based swin transformer algorithm in CMR analysis.

FIGS. 12A and 12B show the schematic overview of the VST-based framework for modeling SAX cine. The developed model may have four stages 1292—e.g., four Video Swin Transformer blocks 1290. Each stage, besides the last stage, may perform 2× spatial downsampling in the patch merging layer. No downsampling may be along the temporal dimension. The patch merging layer 1240 may concatenate the features of each group of 2×2 spatially neighboring patches and may apply a linear layer to project the concatenated features to half of their dimension. The Video Swin Transformer block may have a 3D window based multi-head self-attention module (3D W-MSA) 1092 and a 3D shifted window based multi-head self-attention module (3D SW-MSA) 1294, followed by a feed-forward network—e.g., a two-layer multi-layer perceptron (MLP) 1296, with Gaussian Error Linear Unit (GELU) non-linearity in between. Layer Normalization (LN) 1298 may be applied before each MSA module and MLP, and a residual connection may be applied after each module. The number of heads for each stage may include 4, 8, 16, and 32.

Data may be augmented. Model performance may improve with increasing training data sample size. For the screening model, random rotation, random color jitter, and adding random number may be used. During each step of stochastic gradient descent in the training process, each training sample, cine video sequences may be perturbed with a random rotation (between −45 to +45 degrees for SAX cine; between −20 to +20 degrees for 4CH cine), random color jitter, and with adding a number sampled uniformly between −0.1 to 0.1 to image pixels (pixel values may be normalized) to increase or decrease brightness of the images. For LGE, a random rotation between −45 to +45 degrees, random color jitter, and random flip along z-axis may be used. Data augmentation may result in improvement for all models.

FIG. 12B shows the models may fuse multiple modes. First, VST-based models for SAX cine, 4CH cine, and SAX LGE, may be developed, respectively. Then, to fuse information from different modalities, a global average pooling layer 1250 (FIG. 12A) may be added following the last self-attention module for each VST model. This may result in a 1024-dimension feature vector from each modality. The 1024-dimension vectors may be concatenated and added a fully-connected layer 1260 on top of that to aggregate the features. The final fully connected softmax layer 1260 may produce a distribution over the output classes 1270. In terms of training, the pre-trained weights of each VST branch may be loaded and frozen from different modalities using transfer learning and may be finetuned the last fully-connected layers for feature aggregation.

Many details may be attended to in implementation. Following the classic VST configuration, an AdamW optimizer may be employed using a cosine decay learning rate scheduler and 2.5 epochs of linear warm-up. A batch size of 32 may be used. The backbone VST may be initialized from the ImageNel and Kinetics-600 pre-trained model; the head may be randomly initialized. Model pre-training may play a role in VST-based CMR interpretation. Multiplying the learning rate of the backbone by 0.1 may improve performance. Specifically, the initial learning rates for the pre-trained backbone and randomly initialized head may be set to be 1e-4 and 1e-3, respectively.

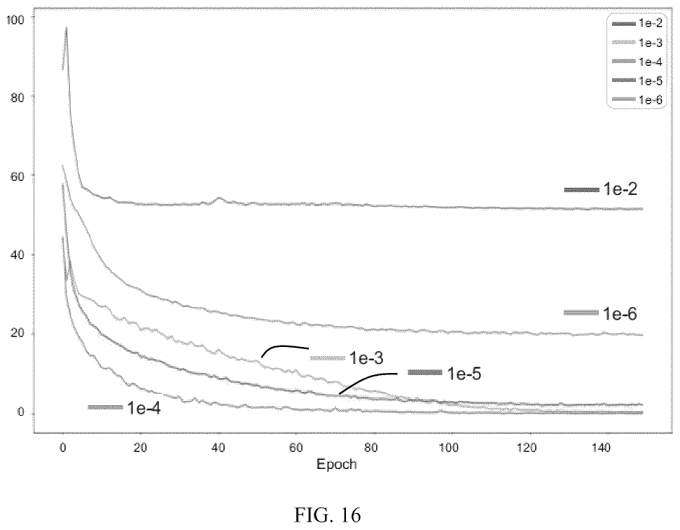

The impact of learning rate modification on the VST backbone was systematically examined as below (FIG. 16). 0.2 stochastic depth rate and 0.05 weight decay for the Swin-Base model may be adopted. To prevent the models from becoming biased towards one class, the training datasets for both screening and diagnostics using ClassBalancedDataset sampling strategy may be balanced. Each VST branch derived from the single modality may be trained for epochs and then may be fed into the fusion model, following with 20 epochs of finetuning particularly for the fusion layers. For inference, the batch size may be set to be one and the number of workers may be four. The training time for model development using four NVIDIA GeForce RTX 3090 GPUs with 24 GB VRAM may be about 77 hours, and the inference time for each subject may be only 0.233 seconds.

FIG. 12B shows the conventional CNN-LSTM (long short-term memory) architecture for comparison to FIG. 12A. The CNN-LSTM may have a DenseNet encoder 1280 with 40 layers and a growth rate of 12 for feature extraction and an LSTM (long short-term memory) for temporal feature aggregation. DenseNet encoder 1280 may include a series of 2D convolutions, including BN 1282, Relu 1284, 1×1 Conv 1286, and 3×3 Conv 1288 with kernel size 1×1 and 4 3×3 and global average pooling to extract the feature vector for each input frame. For LSTM, the feature vector 1252 for each input frame may be fed into the LSTM module 1254 sequentially. LSTM may fuse 1256 the feature vectors and may produce the final classification score 1270 after one fully connected layer 1260. Video-based deep learning models were trained (FIG. 12A). The model architecture was as follows. For models based on cine sequence (input CMR sequence) 1210, a clip of 13 frames from each 25-frame cine video was sampled using a temporal stride of 2 and spatial size of 224×224, resulting in 7×56×56 input 3D tokens. Referring to FIG. 2B and FIG. 12A, the 3D patch partitioning layer 1220 obtains tokens, with each patch/token having a 128-dimensional feature. Linear embedding 1230 may occur. In practice, 3D convolution without overlapping was applied for this tokenization, and the number of output channels was set to be 128 to project the features of each token to a 128-dimension.

For the training configuration of the CNN-LSTM model (FIG. 12B), the stochastic gradient descent (SGD) optimizer with a learning rate of 0.001, a momentum of 0.9, and a weight decay of 0.001 may be adopted. A batch size of 4 may be used for training and 1 may be used for testing. The DenseNet encoder 1280 of the CNN-LSTM model may be initialized from the pre-trained model; the LSTM component may be randomly initialized. Data augmentation, the input scheme, and computational resources may be kept the same as VST models with the only difference: SAX cine inputs are resized to 64×64 due to CNN-LSTM memory constraints. An independent consecutive test set was used to validate the model. To further evaluate the performance of the developed AI model in a real-world clinical setting, this disclosure constructed a fresh independent testing set, having 1000 subjects consecutively admitted to a hospital in 2023. This consecutive testing set was meticulously designed to be unselected, ensuring a representation of the authentic clinical prevalence and encompassing a diverse spectrum of cardiac disease phenotypes.

Evaluation of the AI screening model was performed as follows. From the 1000 consecutively collected subjects, a testing set for the screening model having 961 subjects was formed with complete cine images, including 159 normal individuals and 802 patients with cardiac anomalies. 39 subjects were excluded based on the following criteria: 1) missing SAX cine or 4CH cine sequences (22 subjects); 2) SAX cine with fewer than 5 views (6 subjects); and 3) inadequate imaging quality (11 subjects). Utilizing cine MRI from both SAX and 4CH views, the AI screening model demonstrated exceptional performance on the independent consecutive testing set (n=961; Table 10), achieving an AUC of 0.984 (95% CI 0.977-0.990) and an F1 score of 0.962 (95% CI 0.953-0.972) for cardiac anomaly screening. The sensitivity of 0.946 (95% CI 0.930-0.964) was achieved by the screening model for cardiac anomaly detection with specificity at 90%.

TABLE 10
Distribution of demographics and LVEF across 11 CVD classes
and the normal control class in the primary dataset.

| | | | | | | | | LVEF | |
|---|---|---|---|---|---|---|---|---|---|
| | | No. of | Sex | | | | Age | Mean | Median |
| | | Subjects | Male | | Female | | (Range) | (STD) | (Q1, Q3) |
| | Normal | 1250 | 700 | (56%) | 550 | (44%) | 37 ± 14 | 60.1 (5.9) | 60.0 |
| | Controls | | | | | | (10-78) | | (56.0, 64.0) |
| 1 | HCM | 2327 | 1513 | (65%) | 814 | (35%) | 48 ± 14 | 65.2 (5.8) | 66.0 |
| | | | | | | | (7-86) | | (62.0, 69.0) |
| 2 | DCM | 1435 | 1076 | (75%) | 359 | (25%) | 44 ± 15 | 25.9 (9.1) | 25.0 |
| | | | | | | | (4-82) | | (19.0, 32.0) |
| 3 | CAD | 942 | 829 | (88%) | 113 | (12%) | 56 ± 11 | 34.8 (16.2) | 33.0 |
| | | | | | | | (8-83) | | (24.0, 43.0) |
| 4 | LVNC | 291 | 192 | (66%) | 99 | (34%) | 39 ± 16 | 38.1 (14.8) | 36.0 |
| | | | | | | | (6-77) | | (25.9, 52.0) |
| 5 | RCM | 355 | 170 | (48%) | 185 | (52%) | 50 ± 20 | 53.6 (8.6) | 53.0 |
| | | | | | | | (7-85) | | (48.0, 60.0) |
| 6 | CAM | 220 | 156 | (71%) | 64 | (29%) | 56 ± 11 | 45.7 (11.4) | 47.0 |
| | | | | | | | (18-83) | | (38.1, 54.0) |
| 7 | HHD | 402 | 366 | (91%) | 36 | (9%) | 42 ± 13 | 41.9 (15.2) | 40.9 |
| | | | | | | | (12-75) | | (30.1, 54.0) |
| 8 | Myocar- | 87 | 64 | (74%) | 23 | (26%) | 28 ± 11 | 55.3 (10.5) | 57.0 |
| | ditis | | | | | | (14-69) | | (53.5, 61.0) |
| 9 | ARVC | 370 | 245 | (66%) | 125 | (34%) | 39 ± 14 | 45.8 (13.9) | 48.0 |
| | | | | | | | (9-74) | | (36.0, 56.7) |
| 10 | PAH | 134 | 36 | (27%) | 98 | (73%) | 32 ± 12 | 56.3 (7.2) | 56.0 |
| | | | | | | | (10-72) | | (51.9, 60.1) |
| 11 | Ebstein's | 87 | 33 | (38%) | 54 | (62%) | 34 ± 16 | 53.1 (9.9) | 54.0 |
| | Anomaly | | | | | | (2-63) | | (47.8, 60.0) |

* LVEF: left ventricular ejection fraction.

The screening model performance is detailed in Table 8. Table 8 shows performance of the screening model in the consecutive testing set (n=961). Notably, the consecutive testing set encompassed a diverse range of cardiovascular diseases, including mild/borderline cases and suspected phenocopies (e.g., inherited metabolic cardiomyopathies), extending beyond the commonly identified 11 CVD classes. This underscores the robustness of the screening model with respect to both disease types and severity.

TABLE 8
Performance of the screening model in
the consecutive testing set (n = 961).

| | Screening Model |
|---|---|
| Performance | (SAX + 4CH cine) |
| AUROC | 0.984 (0.977-0.990) |
| PPV | 0.971 (0.957-0.982) |
| Specificity with sensitivity at 90% | 0.994 (0.965-1.000) |
| Sensitivity with specificity at 90% | 0.946 (0.930-0.964) |
| F1-score | 0.962 (0.953-0.972) |

AUROC = area under the receiver operating characteristic curve; PPV = positive predictive value (precision); CI = confidence intervals; SAX = short axis; 4CH = four chamber.

The AI diagnostic model was evaluated as follows. From the 1000 consecutively collected subjects, a testing set for the diagnostic model was formed, having 532 patients with CVD and complete sets of LGE and cine images. To ensure the integrity of the testing set, a detailed exclusion criteria was established. Specifically, 159 normal individuals without cardiac anomalies were excluded, along with 222 patients lacking LGE images, which are essential inputs for the diagnostic model. LGE, an invasive exam requiring contrast injection, wasn't performed for all admitted patients. Additionally, 48 patients with cardiovascular disease, falling beyond the scope of the commonly identified 11 CVD classes, were excluded from the reported quantitative testing performance. Nevertheless, the AI screening and diagnostic results for these 48 patients were included and analyzed.

The AI screening model demonstrated robust performance by correctly classifying all patients into the abnormal class, with a high average confidence score of 0.918. This successful classification, along with the high confidence score, highlights the screening model's robustness in handling a diverse range of cardiovascular diseases, including suspected phenocopies, such as genetic metabolic cardiomyopathy, which extend beyond the commonly recognized 11 CVD classes.

In contrast, the diagnostic model classified these cases with an average low confidence score of 0.585, emphasizing the model's cautious approach when dealing with instances that deviate from the specified 11 CVD classes. An additional AI deferral system could defer cases with low confidence scores, falling below a predefined threshold, for expert human assessment. This collaborative synergy between human clinicians and AI models may further improve diagnostic accuracy, especially in scenarios beyond the commonly specified 11 CVD classes.

In an example in accordance with this disclosure, a method may include selecting a treatment based on the diagnostic prediction, which may further have a step of triaging the radiographic image sequence when an F1 score or confidence in the prediction or a value derived from the cine MRI sequence and the late gadolinium enhancement sequence. In an example, the F1 score or confidence in the prediction may be less than 0.724. In another example, the F1 score or confidence in the prediction may be lower than a threshold value between 0.92 and 0.59. In another example, the F1 score or confidence in the prediction may be a threshold value selected from the range of 0.95 to 0, inclusive.

With the established testing set (n=532), the AI diagnostic model, utilizing cine and LGE images as combined inputs, demonstrated exceptional performance. It achieved a class-weighted average area under the curve (AUC) of 0.986 and an F1 score of 0.903 (Table 9). Table 9 shows performance of the diagnostic model in the consecutive testing set (n=532). Notably, the model exhibited high AUCs and F1 scores for prevalent CVDs, including HCM (AUC: 0.993 [0.988-0.997]; F1: 0.958 [0.940-0.975]), DCM (0.991 [0.983-0.996]; 0.922 [0.883-0.958]), and CAD (0.997 [0.994-0.999]; 0.915 [0.855-0.966]). Across all eleven CVD classes, the model achieved an AUC greater than 0.90, with F1 scores above 0.80 for all except LVNC, HHD, RCM, and myocarditis. The cardiac amyloidosis (CAM) class exhibited a high F1 score of 0.947 and an AUC of 1.0.

TABLE 9
Performance of the diagnostic model in the
fresh consecutive testing set (n = 532).

| | | No. of | Diagnostic Model (cine + LGE) | |
|---|---|---|---|---|
| CVD class | | Subjects | AUROC (95% CI) | F1 score (95% CI) |
| 1 | HCM | 239 | 0.993 (0.988-0.997) | 0.958 (0.940-0.975) |
| 2 | DCM | 107 | 0.991 (0.983-0.996) | 0.922 (0.883-0.958) |
| 3 | CAD | 58 | 0.997 (0.994-0.999) | 0.915 (0.855-0.966) |
| 4 | LVNC | 10 | 0.992 | 0.727 |
| 5 | RCM | 8 | 0.997 | 0.762 |
| 6 | CAM | 10 | 1.000 | 0.947 |
| 7 | HHD | 72 | 0.942 (0.904-0.970) | 0.742 (0.656-1.000) |
| 8 | Myocarditis | 10 | 0.991 | 0.706 |
| 9 | ARVC | 15 | 0.993 | 0.889 |
| 10 | PAH | 0 | — | — |
| 11 | Ebstein's | 3 | 1.000 | 1.000 |
| | Anomaly | | | |

| | | |
|---|---|---|
| Class frequency-weighted | 0.986 | 0.903 |
| average | | |

AUROC = area under the receiver operating characteristic curve; CI = confidence intervals. The calculation of the 95% CI was not performed for sample sizes below 50 due to potential limitations in the precision of estimates associated with small sample sizes.

The application of CMR encompasses virtually all aspects of cardiovascular diseases. It shows unique capabilities in the diagnostic workup of suspected cardiovascular disease. However, CMR is also one of the most challenging radiologic imaging techniques to interpret due to the complexity of cardiac motion. This disclosure shows a pioneering investigation in computerized CMR (cine and LGE) interpretation for screening and diagnostics. This disclosure of 8066 CVD patients and 1653 normal individuals concluded that the screening model for anomaly detection and diagnostic model for CVD classification attained AUCs of 0.988±0.3% and 0.991±0.0% (F1 scores of 0.974±0.5% and 0.895±1.6%; mean+s.d. of internal set and external set), respectively. These results demonstrate that video-based end-to-end deep learning approaches may reliably detect anomalies and classify various types of CVDs from CMR with high classification performance similar to or even superior to that of experienced cardiologists.

This disclosure may show an automatic pathway to CMR analysis. In contrast to manual, conventional clinical approaches, deep neural networks (DNNs) may enable an approach that may be fundamentally different since the automatic model may absorb all pieces of information present in CMR ‘end-to-end’ without requiring manual tracing, calculation of cardiac function, and class-specific feature extraction. In other words, the DNN model may accept the raw CMR data as input, may learn all of the important features, both previously manually derived and as-yet-unrecognized, in a data-driven way, and may output final diagnostic probabilities.

The high performance of the developed screening models derived from cine-MRI May suggest a fast, non-invasive, and accurate screening technique for detecting cardiovascular diseases. The screening model derived from 4CH cine achieved an AUC of 0.977±0.4% (mean±s.d. of internal set and external set; Table 4); the model derived from SAX cine achieved an AUC of 0.962±1.3%. The single view schemes yielded similar performance as combined views (the model derived from 4CH and SAX cine received an AUC of 0.988±0.3%). Therefore, the finding that a single view may independently and reliably detect cardiac anomalies indicates that this method may be used to simplify CMR acquisition and improve clinical efficiency.

Increased efficiency may be beneficial, given the potential to decrease the cost of cine MRI acquisition and enhance patient throughput. The shortened procedure time may be also beneficial for patients who cannot tolerate longer scans, such as pediatric patients. In addition, cine MRI may provide high-resolution images for accurate quantitation of ventricular volume, cardiac function, and motion estimation, along with detailed signals in myocardium, which together may form the cornerstone of diagnosis. As such, the cine-based screening test may improve the accuracy of anomaly detection in CVD, particularly since there is ample evidence to suggest that the most widely used screening exams—electrocardiogram (ECG) and echocardiogram (echo)—capture only a fraction of the informative features for diagnosis. Thus, an instrument that incorporates the cine-based screening method may be an improvement over the most widely used screening instruments.

CVD diagnosis is one of the most problematic and challenging tasks in cardiology. To address the challenge, this disclosure introduced automatic diagnosis based on CMR. Cine and LGE MRIs may be used together to outperform a model derived from either cine or LGE alone. The diagnostic model derived from cine and LGE yielded an average class-weighted AUC of 0.991 over eleven classes. The eleven classes account for most of the cardiovascular diseases referred for CMR examination, making the model applicable to most cardiovascular diseases. This diagnostic model may enable efficient and precise CVD diagnosis that may have a significant clinical impact. The AI model may also expand the capability of a CMR-trained cardiologist in the clinical workflow by triaging the readings for which the model has the least ‘confidence’. For example, when triaging, the model could output only those diagnoses that return the highest confidence values or could not report diagnoses with an F1 score lower than a threshold, for instance where the threshold is a number selected from the range of 0.95 to 0.

Moreover, the AI models may outperform cardiologists in diagnosing PAH by successfully identifying CMR-negative cases (e.g., confirmed PAH without significant abnormal CMR findings that may be indicative of cardiovascular disease). This diagnosis may have marked clinical impact by allowing for less invasive diagnosis of PAH, for example, without a right heart catheterization (RHC). PAH is a progressive condition with high mortality, and timely diagnosis is vital for its treatment. The current convention for diagnosis of PAH is RHC, which is an invasive procedure that can introduce serious surgical complications including hematoma, pneumothorax, arrhythmias, and hypotensive episodes. The diagnosis of PAH may be made based on the processing of the CMR data using machine learning models that analyze relevant imaging biomarkers, including but not limited to, right ventricle size and function, pulmonary artery dimensions, and other associated parameters, thereby enabling the prediction of PAH severity and the potential exclusion of alternative diagnoses, such as chronic obstructive pulmonary disease (COPD) or left heart failure, without the need for invasive diagnostic procedures like RHC. CMR's diagnostic utility in PAH is largely underexplored due to its technical complexity. The AI-empowered CMR interpretation demonstrated in this disclosure may offer a timely and valuable perspective and pathway for an accurate, safe, and rapid PAH diagnosis.

Of the CVD classes examined, myocarditis is a clinically important cardiovascular disease for which the diagnostic model derived from cine and LGE had a lower F1 score compared to other CVD classes (internal set: 0.724; external set: 0.630). Manual review of the discordances revealed that the model misclassifications overall appear very reasonable. For example, some instances of mild myocarditis only present mild elevation of troponin with no remarkable myocardial necrosis, leading to an LGE-negative result. Meanwhile, the edema and functional ventricular impairment may be relieved if patients with myocarditis are not scanned in the appropriate time window, resulting in CMR negativity. The sensitivity of myocarditis diagnosis based on the Lake Louise criteria—the diagnostic CMR imaging criteria for patients with suspected myocarditis—only reaches 0.780-0.875. Moreover, for myocarditis diagnosis, the lack of T2-weighted images and parametric myocardial mapping limited the conclusions that could reasonably be drawn from the cine and LGE MRI, making it more difficult to definitively ascertain whether the cardiologists and/or the AI model was correct.

This disclosure provides a representative CMR dataset covering a wide spectrum of types of CVDs; accounting for above 90% of the CVD patients referred for CMR examination. Additionally, the CMR was acquired by three major vendor instruments. This disclosure represents end-to-end deep learning approaches for screening and diagnostics and comprehensive internal and external validations of 9,717 subjects pooled from eight medical centers. The disclosure leveraged more than one million cardiac MRI images having 38,868 cine films and 72,594 LGE images. Large pooled CMR databases containing both cine and LGE modalities which can be used to diagnose a wide range of heart conditions do not currently exist. As such, the collected cohort is unique in that it is the largest and first-ever complete CMR database with cine and LGE MRIs for artificial intelligence-enabled studies.

Datasets used in this disclosure include eight health centers with identified patients with CVDs and normal controls. All data were anonymized and deidentified, as per the Health Insurance Portability and Accountability (HIPAA) Act Safe Harbor provision. Inclusion criteria included the following: (1) patients with a definitive diagnosis of cardiovascular disease (CVD); (2) patients with CMR scans at baseline before surgical treatment, if any. Exclusion criteria were (1) incomplete cine or LGE modalities; (2) SAX cine with fewer than 5 views; (3) CMR images with insufficient scan quality; (4) CVD patients missing clinical data; (5) CMR exams that could not be interpreted and agreed upon by the committee cardiologists according to diagnostic criteria. Table 10 shows the detailed demographics and distribution of the primary dataset, and the external validation sets collected from the other seven medical centers.

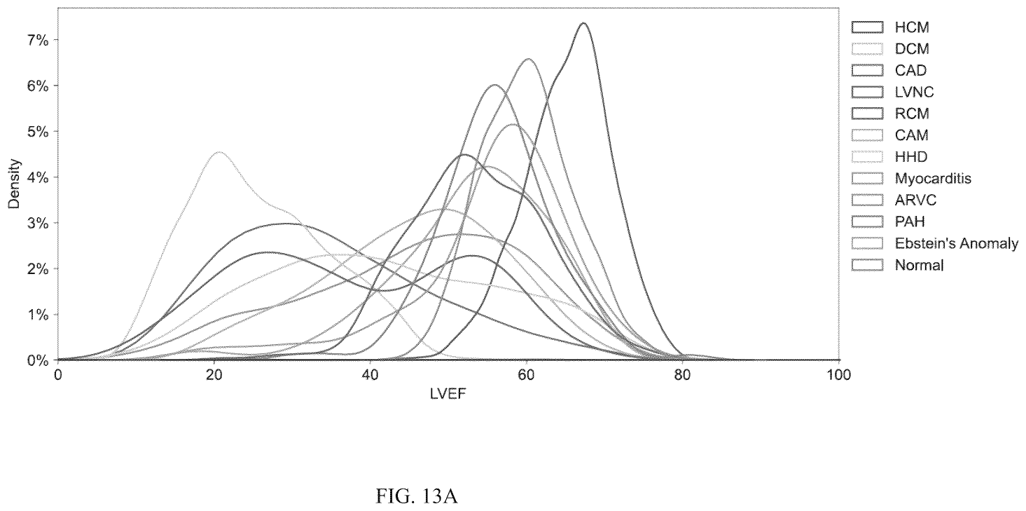

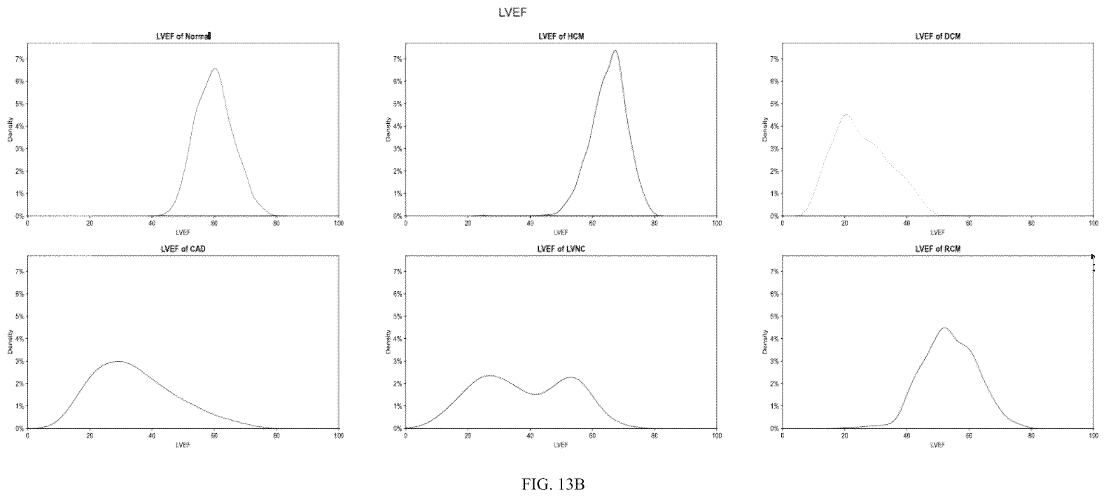

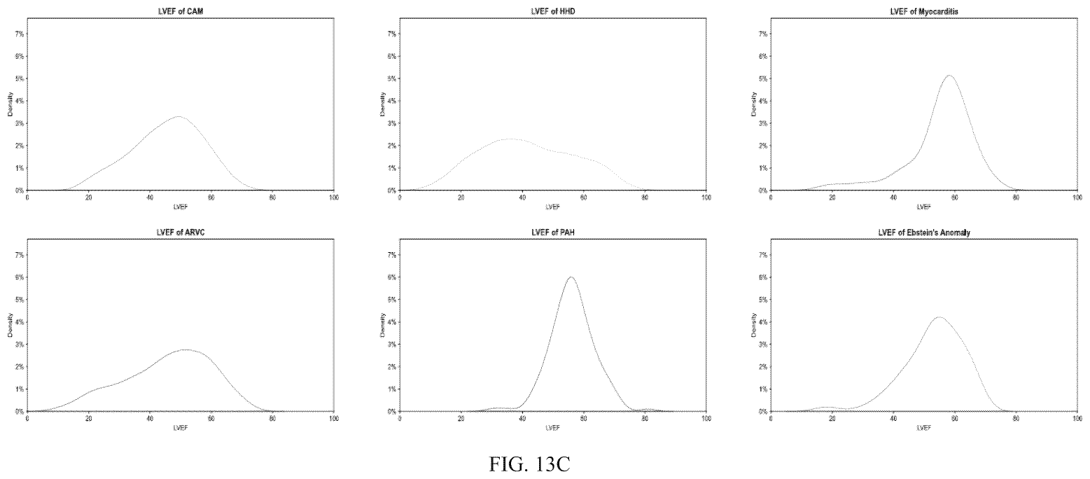

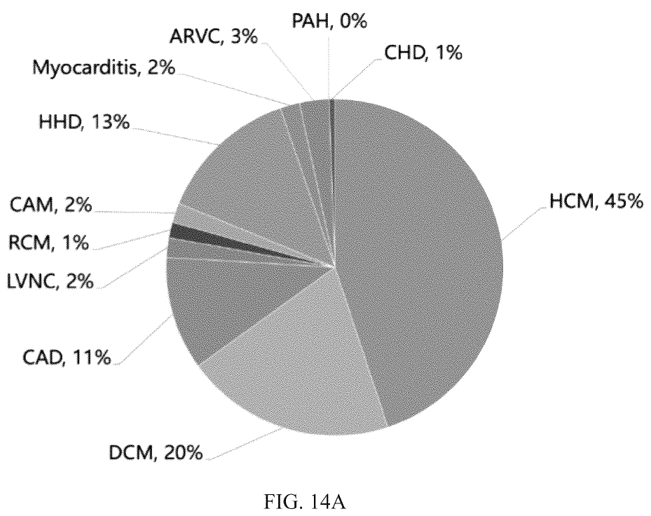

FIGS. 13A-13C show distributions of a characteristic of the screening and diagnostic systems, methods, and device in accordance with examples of the present disclosure. In order to offer a comprehensive perspective on the primary development dataset, the left ventricle ejection fraction (LVEF) metric was collected for all 7900 subjects (including 1250 normal controls and patients with cardiovascular disease) within the primary dataset. The summarized distribution of demographics and LVEF were meticulously summarized across the 11 specified cardiovascular disease classes and the normal control class in Table 10. Table 10 shows distribution of demographics and LVEF in the primary dataset. Additionally, density plots were generated to illustrate the distribution of LVEF for each class in the primary dataset, offering a more comprehensive representation (shown in FIGS. 13A-13C). FIGS. 13A-13C shows the distribution of LVEF across the 11 CVD classes and the normal control class in the primary dataset.

The fresh consecutive testing set is designed to capture the genuine spectrum of disease phenotypes in the real-world clinical prevalence. To offer a thorough understanding of the severity of cases in alignment with real-world clinical prevalence, five key cardiac function parameters, which may be quantitated are presented. These parameters or metrics include LVEF, LV mass (left ventricular mass), LVMi (left ventricular mass index), LVEDV (left ventricular end-diastolic volume), and LVEDVi (left ventricular end-diastolic volume index). Table shows distribution of demographics and the cardiac functions across 11 cardiovascular disease classes and the normal control class in the fresh consecutive testing set and distribution of demographics and cardiac function in the consecutive testing set.

Table 11: Distribution of demographics and cardiac function across 11 cardiovascular disease classes and the normal control class in the independent consecutive testing set

| | | | | | | | | LVEF | | | LV mass | | |
|---|---|---|---|---|---|---|---|---|---|---|---|---|---|
| | | | Sex | | | | Age | Mean | | Median | Mean | | Median |
| | | Number | Male | | Female | | (Range) | (STD) | | (Q1, Q3) | (STD) | | (Q1, Q3) |
| | Total | 691 | 465 | (67%) | 226 | (33%) | 45 ± 16 | 53.5 | (16.3) | 60.0 | 126.8 | (58.6) | 114.0 |
| | | | | | | | (2-86) | | | (41.3, | | | (85.9, |
| | | | | | | | | | | 66.0) | | | 161.0) |
| | Normal | 159 | 83 | (52%) | 76 | (48%) | 37 ± 16 | 63.0 | (5.3) | 63.0 | 77.5 | (25.6) | 72.4 |
| | Controls | | | | | | (11-77) | | | (59.7, | | | (57.6, |
| | | | | | | | | | | 66.3) | | | 94.7) |
| 1 | HCM | 239 | 160 | (67%) | 79 | (33%) | 49 ± 15 | 65.2 | (7.1) | 66.0 | 150.1 | (62.5) | 138.8 |
| | | | | | | | (7-86) | | | (62.0, | | | (102.9, |
| | | | | | | | | | | 70.0) | | | 179.3) |
| 2 | DCM | 107 | 74 | (69%) | 33 | (31%) | 45 ± 15 | 31.3 | (10.1) | 31.0 | 129.9 | (46.5) | 119.9 |
| | | | | | | | (2-77) | | | (22.9, | | | (96.3, |
| | | | | | | | | | | 40.0) | | | 158.2) |
| 3 | CAD | 58 | 51 | (88%) | 7 | (12%) | 53 ± 12 | 35.7 | (13.2) | 33.0 | 129.9 | (44.3) | 121.0 |
| | | | | | | | (29-81) | | | (26.5, | | | (97.5, |
| | | | | | | | | | | 44.5) | | | 155.0) |
| 4 | LVNC | 10 | 7 | (70%) | 3 | (30%) | 35 ± 13 | 45.3 | (12.6) | 47.5 | 104.7 | (42.7) | 100.2 |
| | | | | | | | (17-57) | | | (42.3, | | | (71.8, |
| | | | | | | | | | | 55.5) | | | 123.3) |
| 5 | RCM | 8 | 1 | (12%) | 7 | (88%) | 45 ± 18 | 56.5 | (10.3) | 56.2 | 58.4 | (19.3) | 57.4 |
| | | | | | | | (13-69) | | | (53.0, | | | (47.8, |
| | | | | | | | | | | 61.4) | | | 74.7) |
| 6 | CAM | 10 | 6 | (60%) | 4 | (40%) | 62 ± 10 | 49.9 | (11.2) | 49.1 | 134.1 | (38.3) | 124.5 |
| | | | | | | | (40-73) | | | (42.5, | | | (112.8, |
| | | | | | | | | | | 59.5) | | | 171.5) |
| 7 | HHD | 72 | 64 | (89%) | 8 | (11%) | 43 ± 13 | 44.6 | (13.7) | 42.5 | 168.1 | (60.5) | 158.5 |
| | | | | | | | (16-71) | | | (33.9, | | | (125.3, |
| | | | | | | | | | | 54.3) | | | 203.2) |
| 8 | Myocar- | 10 | 7 | (70%) | 3 | (30%) | 40 ± 19 | 54.1 | (11.7) | 56.5 | 99.8 | (31.1) | 91.0 |
| | ditis | | | | | | (14-70) | | | (46.0, | | | (86.0, |
| | | | | | | | | | | 63.4) | | | 113.4) |
| 9 | ARVC | 15 | 10 | (67%) | 5 | (33%) | 52 ± 13 | 42.3 | (12.4) | 44.7 | 89.6 | (29.0) | 87.2 |
| | | | | | | | (27-67) | | | (35.9, | | | (64.9, |
| | | | | | | | | | | 48.2) | | | 115.7) |
| 10 | PAH | 0 | — | | — | | — | — | | — | — | | — |
| 11 | Ebstein's | 3 | 2 | (67%) | 1 | (33%) | 33 ± 8 | 61.1 | (6.6) | 63.6 | 72.6 | (15.9) | 80.7 |
| | Anomaly | | | | | | (25-41) | | | (58.6, | | | (67.4, |
| | | | | | | | | | | 64.8) | | | 81.7) |

| | | LVMi | | | EDV | | | EDVi | | |
|---|---|---|---|---|---|---|---|---|---|---|
| | | Mean | | Median | Mean | | Median | Mean | | Median |
| | | (STD) | | (Q1, Q3) | (STD) | | (Q1, Q3) | (STD) | | (Q1, Q3) |
| | Total | 68.1 | (30.6) | 61.1 | 187.3 | (91.9) | 160.0 | 100.9 | (47.4) | 86.0 |
| | | | | (46.2, | | | (126.3, | | | (71.6, |
| | | | | 83.2) | | | 219.7) | | | 115.5) |
| | Normal | 42.8 | (11.2) | 41.7 | 138.2 | (33.0) | 133.0 | 76.3 | (13.3) | 74.6 |
| | Controls | | | (34.3, | | | (112.3, | | | (67.4, |
| | | | | 50.1) | | | 158.6) | | | 84.6) |
| 1 | HCM | 82.2 | (32.4) | 75.8 | 144.9 | (40.1) | 141.0 | 79.5 | (20.1) | 78.0 |
| | | | | (58.8, | | | (118.7, | | | (68.6, |
| | | | | 100.5) | | | 164.5) | | | 89.1) |
| 2 | DCM | 69.3 | (22.5) | 66.8 | 300.4 | (113.2) | 280.0 | 161.8 | (62.0) | 148.0 |
| | | | | (53.8, | | | (216.9, | | | (121.0, |
| | | | | 81.4) | | | 363.9) | | | 191.8) |
| 3 | CAD | 68.0 | (21.4) | 62.9 | 248.7 | (83.8) | 231.4 | 131.0 | (43.1) | 123.3 |
| | | | | (51.5, | | | (190.9, | | | (100.5, |
| | | | | 81.8) | | | 312.1) | | | 162.2) |
| 4 | LVNC | 57.4 | (22.5) | 54.9 | 219.8 | (90.3) | 181.2 | 120.7 | (47.6) | 102.3 |
| | | | | (39.8, | | | (160.2, | | | (89.4, |
| | | | | 66.1) | | | 282.0) | | | 144.3) |
| 5 | RCM | 38.2 | (12.6) | 38.0 | 99.1 | (38.9) | 95.4 | 64.9 | (28.6) | 57.9 |
| | | | | (31.8, | | | (75.8, | | | (50.7, |
| | | | | 50.0) | | | 105.7) | | | 70.8) |
| 6 | CAM | 88.2 | (37.1) | 75.5 | 118.6 | (35.4) | 121.4 | 74.6 | (18.1) | 82.8 |
| | | | | (66.9, | | | (89.7, | | | (68.0, |
| | | | | 99.5) | | | 145.6) | | | 85.3) |
| 7 | HHD | 84.4 | (34.0) | 77.3 | 236.2 | (93.5) | 225.6 | 117.4 | (48.5) | 108.6 |
| | | | | (60.0, | | | (175.5, | | | (86.5, |
| | | | | 100.1) | | | 263.4) | | | 138.3) |
| 8 | Myocar- | 54.1 | (16.7) | 53.3 | 160.2 | (39.0) | 157.7 | 84.8 | (18.7) | 87.6 |
| | ditis | | | (39.8, | | | (128.3, | | | (74.4, |
| | | | | 63.3) | | | 186.2) | | | 98.9) |
| 9 | ARVC | 49.2 | (13.4) | 47.6 | 204.6 | (66.0) | 220.3 | 113.3 | (33.5) | 116.6 |
| | | | | (37.4, | | | (162.3, | | | (88.4, |
| | | | | 56.9) | | | 232.9) | | | 123.6) |
| 10 | PAH | — | | — | — | | — | — | | — |
| 11 | Ebstein's | 41.7 | (6.7) | 43.6 | 125.0 | (19.3) | 134.7 | 72.8 | (13.4) | 74.3 |
| | Anomaly | | | (39.0, | | | (118.7, | | | (66.5, |
| | | | | 45.4) | | | 136.1) | | | 79.8) |

*Q1: the first quartile; Q3: the third quartile; STD: standard deviation; LV: left ventricular mass; LVMi: left ventricular mass index; EDV: end-diastolic volume; EDVi: end-diastolic volume index; LVEF: left ventricular ejection fraction.

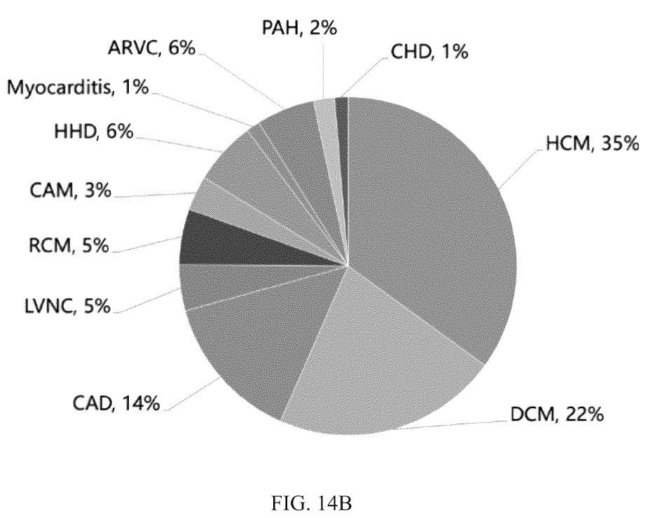

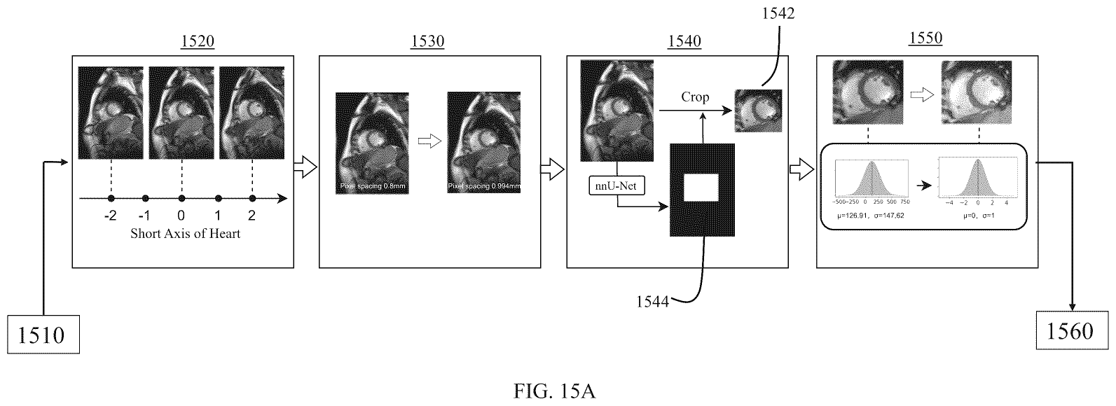

FIGS. 14A and 14B show clinical prevalence of CVD classes in accordance with examples of the present disclosure. For improved visualization and clarity, the prevalence of the eleven CVD classes in both the fresh consecutive testing set (FIG. 14A) (n=532 patients with CVD) and the primary discovery dataset (FIG. 14B) (n=6650 patients with CVD) were deciphered using pie charts. The fresh consecutive testing set FIG. 14A offers a representation of the genuine clinical prevalence. Through direct comparison, it is evident that the primary dataset FIG. 14B and the consecutive testing set FIG. 14A exhibit very similar CVD prevalence and distribution. The top three most prevalent CVDs referred to the CMR examination remain HCM, DCM, and CAD.

All images were acquired by breath-holding and electrocardiogramg. A balanced steady-state free precession (bSSFP) sequence was used for cine images with a continuous sampling from the basal to the apical levels on short-axis views and two-, three-, and four-chamber long-axis views. Cine MRI was included from two views in this data: the standard short-axis (SAX) cine and the long-axis four-chamber (4CH) cine. The SAX cine clearly depicts the right ventricle (RV) and the left ventricle (LV). The 4CH cine shows the four chambers of heart: right atrium, left atrium, right ventricle, left ventricle.

Late gadolinium enhancement (LGE) MRI images were obtained using phase-sensitive inversion recovery (PSIR) sequence with a segmented FLASH readout scheme performed 10-15 minutes after injection of gadolinium-based contrast with 0.15 mmol/kg per bolus. Gadolinium contrast agents can be used to detect areas of fibrosis, as the prolonged washout of the contrast correlates with a reduction in functional capillary density in the irreversibly injured myocardium. The SAX LGE used in the disclosure was acquired from the short-axis view with the same section thickness, covering the entire left ventricle from the base to the apex (9 parallel views for most cases). Note that LGE is an invasive exam that requires contrast injection and was therefore not performed for normal controls.

An example CMR scan protocol and scanner parameters for the primary and external validation sets is shown in Table 12. Table 12 shows an example CMR scan protocol and scanner parameters for the primary and external sets. FIG. 8 shows an illustration of cardiac MRIs (SAX cine, 4CH cine, SAX LGE) utilized in model development.

TABLE 12
The typical CMR scan protocol and scanner parameters for the primary and external sets.

| | | FW | | | AZ | GD | HEB | LZ | RJ | TJ | XH |
|---|---|---|---|---|---|---|---|---|---|---|---|
| | | Manufacture | | | | | | | | | |
| | | | GE | | | | | | | | |
| | | SIEMENS | Healthcare | Philips | Philips | Philips | Philips | Philips | Philips | SIEMENS | SIEMENS |
| Magnetic | | 3 | 3 | 3 | 3 | 3 | 3 | 3 | 3 | 3 | 3 |
| field strength | | | | | | | | | | | |
| CINE | Slice thickness | 8 | 8 | 8 | 8 | 8 | 8 | 8 | 6 | 8 | 8 |
| | (mm) | | | | | | | | | | |
| | Slice spacing | 10 | 8 | 10 | 8 | 10 | 10 | 8 | 6 | 10 | 10 |
| | (mm) | | | | | | | | | | |
| | Typical field | 35 | 35 | 35 | 27 | 24 | 30 | 35 | 30 | 36 | 35 |
| | of view (cm) | | | | | | | | | | |
| | Echo time | 1.47 | 1.69 | 1.48 | 1.60 | 1.50 | 1.50 | 1.60 | 1.60 | 1.42 | 1.41 |
| | (ms) | | | | | | | | | | |
| | Temporal | 43.42 | 53.28 | 47.4 | 49.00 | 44.00 | 67.00 | 49.00 | 80.00 | 37.68 | 45.08 |
| | resolution (ms) | | | | | | | | | | |
| | Flip angle | 52 | 50 | 45 | 45 | 45 | 45 | 45 | 45 | 46 | 50 |
| | (degrees) | | | | | | | | | | |
| | Pixel Bandwidth | 990 | 488 | 1701 | 2164 | 1420 | 2188 | 1938 | 1827 | 965 | 960 |
| | (Hz/pixel) | | | | | | | | | | |
| LGE | Slice thickness | 8 | 8 | 8 | 8 | 8 | 8 | 8 | 10 | 8 | 8 |
| | (mm) | | | | | | | | | | |
| | Slice spacing | 9.6 | 8 | 9 | 8 | 10 | 10 | 8 | 10 | 10 | 10 |
| | (mm) | | | | | | | | | | |
| | Typical field | 38 | 35 | 36 | 27 | 25 | 30 | 35 | 30 | 34 | 35 |
| | of view (cm) | | | | | | | | | | |
| | Echo time | 1.96 | 2.78 | 3 | 3.00 | 3.00 | 3.00 | 3.00 | 3.00 | 1.20 | 2.00 |
| | (ms) | | | | | | | | | | |
| | Repetition | 6 | 5.98 | 6 | 6.06 | 6.13 | 6.10 | 6.10 | 6.10 | 6 | 6 |
| | time (ms) | | | | | | | | | | |
| | Inversion | 300 | 300 | 300 | 300 | 300 | 300 | 350 | 375 | 280 | 360 |
| | Time (ms) | | | | | | | | | | |
| | Flip angle | 20 | 25 | 25 | 25 | 25 | 25 | 25 | 25 | 55 | 20 |
| | (degrees) | | | | | | | | | | |
| | Pixel Bandwidth | 285 | 244 | 250 | 226 | 257 | 258 | 253 | 253 | 770 | 285 |
| | (Hz/pixel) | | | | | | | | | | |

FW: Beijing Fuwai Hospital, Beijing; AZ: Beijing Anzhen Hospital, Beijing; GD: Guangdong Provincial People's Hospital, Guangzhou; HEB: The 2nd Affiliated Hospital of Harbin Medical University, Harbin; LZ: The First Hospital of Lanzhou University, Lanzhou; RJ: Renji Hospital, Shanghai; TJ: Tongji hospital, Wuhan; XH: Peking Union Medical College Hospital, Beijing.

The datasets were annotated as follows. For each patient in the disease cohort, the textual description of the abnormalities in the CMR and the clinical report was extracted as the main reference. Besides that, all CMR records underwent additional annotation procedures. To annotate the disease cohort, a group of certified CMR experts reviewed all records and clinical reports. Every record was randomly assigned to be reviewed by a single physician specifically for this task, not for any other purpose. All annotators received specific instructions and training regarding how to annotate CMR data to improve labeling consistency. CMR exams that could not be interpreted by physicians received further annotation from a consensus committee of board-certified practicing cardiologists (with >15 years of experience in CMR reading) working in a hospital. The CMR exams that could not be interpreted or agreed upon by the committee were removed from our dataset.

For the independent conventional-standard test dataset with 500 patients for human-machine comparison, six physicians working in the magnetic resonance imaging department at a hospital contributed directly to its annotation. The six physicians were not involved in dataset annotation as described above. All participating physicians received specific instructions and training regarding how to annotate CMRs to ensure consistency. The physicians were divided into three groups according to their reading experience in CMR: 3-5 years, 5-10 years, and more than 10 years. CMR physicians in each group reviewed a randomly selected set of the 500 CMRs in a non-repetitive manner.

Referencing FIG. 8, short-axis cine (SAX cine) 810 included 9 parallel views 812 (for most cases) covering the apical to the basal levels of the left ventricle. Each view contained 25 frames (cardiac phases), leading to 325 images in one single SAX cine record. The disclosure shows the representational power of different numbers of input views in developing the classification model. Balancing efficiency and effectiveness, the three-view input scheme achieved a greater representation of SAX cine and thereby was adopted throughout. The three-view input scheme includes the middle layer 814 (the mid slice among the parallel layers spanning from the base to the apex), the second layer above the middle layer 816, and the second layer below the middle layer 818 (FIG. 8).

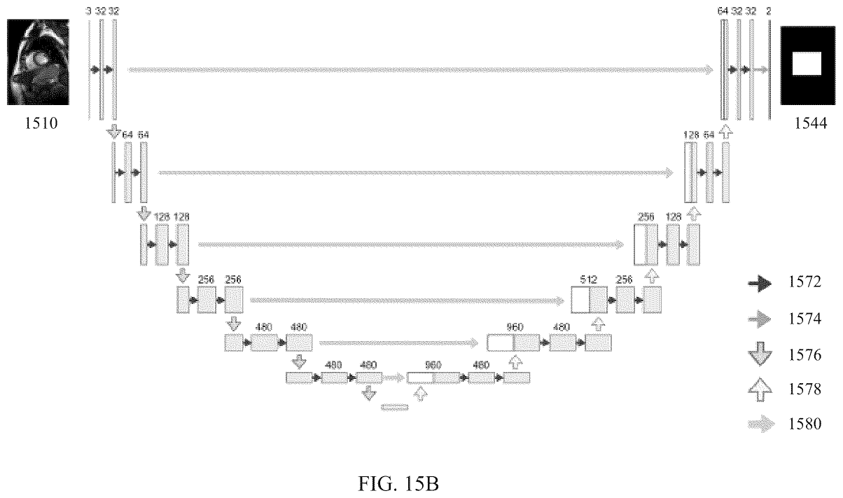

FIGS. 15A and 15B show a preprocessing step for the screening and diagnostic systems, methods, and device in accordance with examples of the present disclosure. Preprocessing May be performed in different sequences of steps. For example, a crop step may come first, and then the rest of the preprocessing steps may follow. In another example, CMR data may be preprocessed as follows. Referencing FIG. 15A, the CMR pre-processing pipeline aimed to remove the additional burden of the deep neural network learning methods to find patterns between images for disease classification. All cardiac MRIs were preprocessed to: (1) resample MRI images to the same spatial resolution 1530; and (2) localize the heart region of interest (ROI) 1540 to a crop image 1542. The disclosure details the preprocessing step for cine and LGE MRI and in FIGS. 15A and 15B.

Referencing FIG. 15A, an image acquisition 1510 is an input into preprocessing. The disclosure shows an extract of the “ImagePositionPatient” tag and the “ImageOrientationPatient” tag from each Dicom header to locate the three layers 1510. Three-spline interpolation 1520 provided by SimpleITK library (https://simpleitk.org/) was applied to re-sample the raw cine MRIs to the same spatial resolution: 0.994 mm×0.994 mm, which is the most common spatial resolution across all subjects investigated. A heart ROI segmentation 1540 model (described in FIG. 15B) was used to localize the region of heart 1542 for each cine MRI. The heart ROI segmentations 1540 predicted by the AI models were manually checked to ensure their accuracy. The extracted ROIs 1540 were padded to keep the aspect ratio the same without distortion, and then resized to 224×224. The top and bottom 0.1% of the pixels in cine MRI images were clipped to avoid pixels that are outliners of the distribution. The cine images were scaled between 1 and 255, and then normalized 1550 by zero-mean and unit-variance before feeding them to the model. The output 1560 is screening and diagnostic classification.

The disclosure shows a sample clip of 25 frames from each full length cine sequence using a temporal stride of two, resulting in 13 frames as inputs to model development. The 4CH cine shares the same pre-processing pipeline as SAX cine, except that only one single layer (mid slice) was used to represent the 4CH view. For SAX LGE, all layers covering from the base to the apex of the heart were used for diagnostic model development. The preprocessing steps for SAX LGE were similar to that of cine MRI. The SAX LGE was resampled along z-axis to ensure that each LGE sequence contains nine slices because nine is the most common number of views for SAX LGE included.

A heart region of interest 1542 was extracted. Heart detection DNN models were used to automatically extract the heart ROI regions 1542. Three DNN models for SAX cine, 4CH cine and SAX LGE were trained and evaluated, respectively. nnU-Net was applied as a model backbone and generated the segmentation masks 1544 for model supervision using a semi-automatic approach: 1). Automatic localization: For SAX cine and 4CH cine, the pixel region was selected with maximum standard deviation across all frames. These regions localize the heart ROI as heart is a beating organ with high standard deviation in its position. Specifically, for each cine movie sequence s={x_1, . . . , x_n}, a single pixel map of standard deviations was computed across all frames x_std=σ({x_1, . . . , x_n}). This map was used to compute an Otsu threshold to binarize and label regions with the greatest variation in cine modality. For each cine sequence, a binary segmentation mask of the heart ROI was defined for the length of the cardiac cycle. All segmentation masks went through manual checking. The localization procedure captured the heart ROI in around 90% of cases. The rest of the cases were labelled manually. 2). Manual labelling: the bounding box was manually drawn capturing the heart ROI, using 3D Slicer and ITK-SNAP. The Scissors tool provided by the Segment Editor in 3D Slicer and the Polygon Inspector in ITK-SNAP was used to locate heart ROI. A binary segmentation mask 1544 was saved for each CMR sequence. For SAX LGE, the annotations were manually drawn as model supervision.

FIG. 15B shows model architecture for generating the segmentation masks 1544. In terms of model architecture, the detection model shares a U-net backbone with three adjustments: 1). batch normalization was replaced with instance normalization; 2). ReLU was replaced with leaky ReLU as the activation function; 3). additional auxiliary losses were added in the decoder to all but the two lowest resolutions. The model may output the binary bounding box 1544 that extracts the heart ROI 1542. FIG. 15B describes process steps in the model, including: Conv 3*3+Instance Normalization+Leaky ReLU 1572; Conv 1*1+Softmax 1574; MaxPooling, Downsample 1576; TransposeConv 2*2, Upsample 1578; and Copy and Concatenate 1580.

For model training, Adam optimizer and stochastic gradient descent (SGD) with Nesterov momentum (μ=0.99) was adopted. The initial learning rate was set to be 0.01, and the decay of the learning rate follows the ‘Poly’ learning rate policy. Batchsize was set to 36. Data augmentation included rotations, scaling, gamma correction, and mirroring. The loss function was the sum of cross-entropy and Dice loss.

In FIG. 15B, the numbers may represent the number of feature channels (or filters) at different stages of the model's encoder-decoder structure. For example, in a typical U-Net architecture for cardiac region segmentation, the numbers 32, 64, 128, 256, 480, and 960 may represent the number of feature channels (or filters) at different stages of the model's encoder-decoder structure. In greater detail, in one example, the encode path or contracting path may include:
1. 32 Channels:
   1. The initial convolution layer may start with 32 feature channels.
   2. This layer captures low-level spatial features from the input image.
2. 64 Channels:
   1. After the first downsampling operation (typically max pooling), the number of channels may double to 64.
   2. This allows the model to learn more complex features as the spatial resolution decreases.
3. 128 Channels:
   1. Another downsampling step increases the number of channels to 128.
   2. The model further abstracts spatial information while deepening feature representation.
4. 256 Channels:
   1. Following another downsampling, the channels increase to 256.
   2. At this stage, the features are more abstract and represent more complex patterns in the cardiovascular structures.
      The bottleneck, or deepest layer may include:
5. 480 Channels:
   a. Before reaching the bottleneck, the model transitions to 480 channels.
   b. This stage may capture the most detailed and abstract features.
6. 960 Channels:
   c. At the bottleneck, the model may achieve its maximum depth with 960 channels.
   d. Here, the model processes highly compressed and representative features of the cardiac regions.
      In a similar example, the decoder path or expanding path may include:
The decoder mirrors the encoder, using transposed convolutions (or upsampling) to gradually restore the spatial dimensions while reducing the number of channels.
The number of channels may halve at each upsampling stage (960→480→256→128 →64→32), while concatenating with corresponding layers from the encoder path to maintain spatial context.

FIG. 16 shows characteristics of learning rate modification of the screening and diagnostic systems, methods, and device in accordance with examples of the present disclosure. The impact of learning rate modification on the VST backbone was systematically examined through a controlled experiment. The experiment encompassed a range of learning rates, from 1e-2 to 1e-6, with a focus on their effects on the AI diagnostic model based on short-axis cine (SAX). AUROC and F1 score for each CVD at learning rates of 1e-3 to 1e-6 is listed in Table 13. The investigation was conducted on the primary cohort (6650 CVD patients), utilizing a two-fold configuration for training and the remaining fold for testing. The model was trained for 150 epochs with five different learning rate initializations for the model backbone: 1e-2, 1e-3, 1e-4 (as applied), 1e-5, and 1e-6, and the training loss for each learning rate is shown in FIG. 16. Other configurations were kept consistent for a fair and direct comparison, and the training loss for each scheme was plotted for analysis. FIG. 16 shows the impact of learning rate modification may have on the VST backbone. When the learning rate is set too high (1e-2; curve), the model may struggle to converge, and the training loss may fail to descend, in stark contrast to the more optimal setting of 1e-4 (curve in green color). The model under the 1e-2 learning rate incorrectly classified all samples into the Hypertrophic Cardiomyopathy (HCM) class during testing. Conversely, when the learning rate is set too low (1e-6; curve in purple color), the loss May descend very slowly over the training period. As depicted in FIG. 16, the loss curves for 1e-5 and 1e-6 remained at a relatively high level compared to the more effective setting of 1e-4.

TABLE 13
The effect of modifying the initialized learning-
rate (testing in on-fold of the primary cohort
with the diagnostic model derived from SAX cine).

| Initialized | AUROC | | | | F1 score | | | |
|---|---|---|---|---|---|---|---|---|
| learning rate | 1e−3 | 1e−4 | 1e−5 | 1e−6 | 1e−3 | 1e−4 | 1e−5 | 1e−6 |
| HCM | 0.992 | 0.989 | 0.990 | 0.987 | 0.941 | 0.945 | 0.937 | 0.914 |
| DCM | 0.973 | 0.975 | 0.972 | 0.962 | 0.825 | 0.849 | 0.817 | 0.788 |
| CAD | 0.959 | 0.962 | 0.949 | 0.901 | 0.747 | 0.757 | 0.728 | 0.589 |
| LVNC | 0.961 | 0.942 | 0.971 | 0.939 | 0.640 | 0.690 | 0.660 | 0.494 |
| RCM | 0.955 | 0.977 | 0.977 | 0.941 | 0.701 | 0.767 | 0.723 | 0.492 |
| CAM | 0.975 | 0.970 | 0.988 | 0.975 | 0.771 | 0.823 | 0.750 | 0.633 |
| HHD | 0.942 | 0.913 | 0.931 | 0.906 | 0.632 | 0.595 | 0.631 | 0.489 |
| Myocarditis | 0.936 | 0.967 | 0.980 | 0.943 | 0.367 | 0.490 | 0.510 | 0.432 |
| ARVC | 0.966 | 0.986 | 0.974 | 0.942 | 0.692 | 0.778 | 0.733 | 0.597 |
| PAH | 0.986 | 0.994 | 0.999 | 0.996 | 0.932 | 0.944 | 0.956 | 0.850 |
| Ebstein's | 0.990 | 0.986 | 0.960 | 0.969 | 0.698 | 0.742 | 0.814 | 0.657 |
| Anomaly | | | | | | | | |
| Class | 0.974 | 0.974 | 0.974 | 0.956 | 0.813 | 0.834 | 0.815 | 0.736 |
| frequency- | | | | | | | | |
| weighted | | | | | | | | |

Further evaluation included the calculation of F1 and AUROC scores for the testing fold under the aforementioned experimental settings (FIG. 16). The model trained with a learning rate of 1e-2 failed to converge and was consequently excluded from the quantitative metrics. According to the evaluation results, the initialized learning rate of 1e-4 may demonstrate superior performance compared to the other settings.

The performance of the AI models may be evaluated quantitatively and statistically by assessing their sensitivity, specificity, precision, and F1 score (harmonic mean of the predictive positive value and sensitivity), with two-sided 95% confidence intervals (CIs), as well as the area under the curve (AUC) of the receiver operating characteristic (ROC) with two-sided CIs. The F1 score may be complementary to the AUC, which is useful in the setting of multi-class prediction and less sensitive than the AUC in settings of class imbalance. For an aggregate measure of model performance, the class frequency-weighted mean for the F1 score and the AUC may be computed.

The cutoff value was set to 0.5 for screening; the CVD class with the highest probability was identified as the diagnostic prediction. In addition, to improve the model interpretability and visualize the features used by the DNN model that determine the final prediction, gradient-weighted class activation mapping (Grad-CAM) may be used to localize important regions-saliency regions—by visualizing class-specific gradient information. After computing the neuron importance weights for each feature map, a heatmap indicating the significant regions related to class c may be generated by performing a weighted linear combination of the feature maps, followed with a ReLU (Rectified Linear Unit) activation.

Then, the Shapley values may be used to evaluate the influence of each input modality (SAX cine, 4CH cine, and SAX LGE). The Shapley value may be a principled attribution method used in artificial intelligence to quantify the contribution of individual input features by assigning each input modality an importance value for a particular prediction.

Coronary artery diseases evaluated include Coronary Artery Disease (CAD)/Ischemic Cardiomyopathy, Hypertrophic Cardiomyopathy (HCM), Dilated cardiomyopathy (DCM), Left Ventricular Non-Compaction Cardiomyopathy (LVNC), Arrhythmogenic right ventricular cardiomyopathy (ARVC), Cardiac amyloidosis, Restrictive cardiomyopathy (RCM), Pulmonary Arterial Hypertension (PAH), Congenital Heart Disease-Ebstein's anomaly, Acute myocarditis, and Hypertensive heart disease (HHD).

With regards to normal controls, healthy controls were recruited as volunteers without cardiovascular diseases (including cardiomyopathy, coronary artery disease, severe arrhythmia/conduction block, valvular disease, and congenital heart disease, etc.) and other organic/systemic diseases on the comprehensive evaluation by patient history, clinical assessment, ECG, and echocardiography.

In summary, this disclosure demonstrates that end-to-end video-based deep learning models can detect cardiac anomalies and further classify distinct cardiovascular diseases from CMR with high classification performance. This disclosure has the potential to substantially advance the efficiency and scalability of CMR interpretation, paving the way for widespread use of CMR in CVD screening and diagnosis.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A computer-implemented method for automated interpretation of cardiac magnetic resonance (CMR) imaging, comprising steps of:
acquiring a sequence of radiographic images of a cardiovascular system, and wherein the sequence of radiographic images is at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence;
processing the sequence of radiographic images using one or more machine learning models, wherein at least one of the one or more machine learning models is a deep learning model;
generating a diagnostic prediction using the one or more machine learning models, wherein the diagnostic prediction is at least one of: a screening assessment of a cardiac anatomy, a diagnostic identification of a cardiovascular condition, a quantitative evaluation of a cardiac function parameter, a structured radiographic report, a natural language summary, or a diagnostic rationale; and
outputting the diagnostic prediction to at least one of: an electronic user interface, a cloud-based platform, a mobile application, a picture archiving and communication system, or an electronic health record.

2. The method of claim 1, wherein acquiring the sequence of radiographic images of the heart further comprises extracting a heart region from the sequence of radiographic images.

3. The method of claim 1, the diagnostic prediction further comprising a classification of the cardiovascular condition, wherein the cardiovascular condition is at least one of: an ischemic heart disease, a nonischemic cardiomyopathy, a pulmonary hypertension, a congenital heart disease, a valvular heart disease, a pericardial disease, an aortic disease, a heart failure syndrome, a myocardial abnormality, an endocardial abnormality, a rhythm disorder, a rare cardiovascular conditions, and a post-treatment cardiac condition.

4. The method of claim 3, wherein the rare cardiovascular condition is at least one of a cardiac tumor, a congenital coronary anomaly, a fabry disease, or a marfan syndrome-related cardiac involvement.

5. The method of claim 3, wherein generating the diagnostic prediction using the one or more machine learning models comprises sequentially generating the screening prediction of the cardiac anatomy and the classification of the cardiovascular condition, and wherein the one or more machine learning models is at least one of: a single multitask neural network architecture, a cascading output model, a hybrid model, or an ensemble model.

6. The method of claim 3, wherein generating the diagnostic prediction using the one or more machine learning models comprises simultaneously generating the screening prediction of the cardiac anatomy and the classification of the cardiovascular condition, and wherein the one or more machine learning models is at least one of: a single multitask neural network architecture, a cascading output model, a hybrid model, or an ensemble model.

7. The method of claim 3, wherein the cardiovascular condition further comprises at least one of a hypertrophic cardiomyopathy, a dilated cardiomyopathy, a coronary artery disease, a left ventricular non-compaction cardiomyopathy, a restrictive cardiomyopathy, a cardiac amyloidosis, a hypertensive heart disease, a myocarditis, an arrhythmogenic right ventricular cardiomyopathy, a pulmonary arterial hypertension, or a congenital heart disease.

8. The method of claim 1, further comprising:
identifying a CMR-negative case; and
diagnosing a patient with a pulmonary arterial hypertension disease without a right heart catheterization of the patient.

9. The method of claim 1, wherein processing the sequence of radiographic images further comprises:
dynamically adjusting the sequence of radiographic images based on an availability of a contrast-enhanced sequence; or
selecting and using at least one of the cine MRI, the T1 mapping, the T2 mapping, the perfusion imaging, the flow quantification, the dark blood imaging, the real-time imaging, the magnetic resonance spectroscopy, or the parametric mapping sequences when the contrast-enhanced sequence is unavailable.

10. The method of claim 1, wherein the one or more machine learning models further comprises at least one of: a video-based swin transformer, a convolutional neural network, a transformer-based model, a CNN-transformer model, a CNN-transformer hybrid model, a vision-language hybrid model, a large language model, a recurrent neural network, a generative adversarial network, a graph neural network, a multi-modal model, a self-supervised learning model, a semi-supervised learning framework, an attention-based model, a reinforcement learning model, or a model that integrates patient data with imaging data.

11. The method of claim 1, wherein the quantitative assessment of the cardiac function parameter is at least one of: a left ventricular ejection fraction, a right ventricular ejection fraction, a wall thickness, a cardiac output, an end-diastolic volume, a systolic volume, an end-diastolic volume index, a stroke volume, a wall motion index, a myocardial strain, a myocardial perfusion, a tissue characterization, a right ventricular volume, a left ventricular volume, a cardiac workload, a myocardial workload, a ventricular mass, a left atrial volume, a right atrial volume, or a left ventricular outflow tract velocity.

12. The method of claim 1, wherein the structured radiographic report is at least one of: a diagnostic impression, a functional metric, a hemodynamic assessment, a morphological characteristic, a myocardial fibrosis marker, a motion analysis, a treatment recommendation, a risk stratification assessment, a disease progression evaluation, a comparison with a prior imaging study, or a summary.

13. The method of claim 1, wherein the one or more machine learning models is deployed on at least one of: a cloud-based application programming interface, an on-premise hospital server, a picture archiving and communication system, a radiology information system, an edge device for point-of-care diagnostics, a mobile platform, a distributed computing environment, or a federated learning framework.

14. The method of claim 1, wherein the diagnostic rationale is at least one of: a visual overlay, an interactive interpretability feature, a visual map highlighting a relevant region of the radiographic image sequence, an image overlay, a segmentation mask, a saliency map, an attention-based visualization, a textual explanation derived from a model parameter or a model activation, a natural language justification generated using a language model, a piece of evidence derived from the radiographic image sequence, a cardiac function, an exclusion of an alternative condition, a confidence score, a cardiac function assessment, or a clinical pathway suggestion.

15. A computer-implemented method for automated diagnosis of cardiovascular diseases using a two-stage deep learning pipeline, comprising steps of:
acquiring a cine MRI sequence of radiographic images of a cardiovascular system without a contrast agent;
processing the cine MRI sequence of radiographic images using one or more machine learning models, wherein at least one of the one or more machine learning models is at least one of a video-based transformer, a convolutional neural network, a recurrent neural network, a transformer-based model, or a multi-modal hybrid architecture;
detecting at least one of a cardiac anomaly, anatomical variation, or a functional abnormality using the cine MRI sequence of radiographic images in a first stage; and
generating a diagnostic classification using the cine MRI sequence of radiographic images and at least one of a late gadolinium enhancement MRI, a T1 mapping, a T2 mapping, a perfusion imaging, a flow quantification, a dark blood imaging, a real-time imaging, a magnetic resonance spectroscopy, or a parametric mapping sequence, in a second stage.

16. The method of claim 15, wherein the cine MRI sequence of radiographic images is at least one of: a short-axis view, a long-axis view, a four-chamber view, a three-chamber view, or a selected representative slice.

17. A computerized system for automated interpretation of cardiac magnetic resonance imaging, the system comprising:
a computerized device having:
a non-transitory memory;
one or more processing apparatuses in communication with the non-transitory memory;
a computer readable storage medium;
a magnetic resonance imaging (MRI) machine in communication with the computerized device, the MRI machine configured to acquire a sequence of radiographic images of a cardiovascular system, wherein the sequence of radiographic images is at least one of: a cine MRI, a late gadolinium enhancement, a T1 mapping, a T2 mapping, a perfusion imaging, a flow quantification, a dark blood imaging, a real-time imaging, a magnetic resonance spectroscopy, or a parametric mapping sequence;
one or more programs comprising program instructions stored on the computer readable storage medium and executable by the one or more processing apparatus via the non-transitory memory, the instructions comprising:
processing the sequence of radiographic images using one or more machine learning models, wherein at least one of the one or more machine learning models is a video-based transformer model, a convolutional neural network (CNN), a recurrent neural network (RNN), a deep neural network (DNN), a graph-based neural network, or a model capable of processing at least one of sequential data or spatiotemporal data;
generating a diagnostic prediction using the one or more machine learning models, wherein the diagnostic prediction is at least one of a screening assessment of a cardiac anatomy, a diagnostic identification of a cardiovascular condition, a quantitative evaluation of a cardiac function parameter, a structured radiographic report, a natural language summary, or a diagnostic rationale; and
outputting the diagnostic prediction to at least one of an electronic user interface, a picture archiving and communication system, a cloud-based platform, a mobile application, or an electronic health record.

18. The system of claim 17, wherein processing the sequence of radiographic images further comprises extracting a region of interest from the sequence of radiographic images.

19. The system of claim 17, wherein generating the diagnostic prediction using the one or more machine learning models further comprises sequentially generating the screening prediction of the cardiac anatomy and a classification of the cardiovascular condition, and wherein the one or more machine learning models is at least one of: a single multitask neural network architecture or a cascading output.

20. The system of claim 17, wherein the diagnostic suggestion of a cardiovascular condition is at least one of a hypertrophic cardiomyopathy, a dilated a cardiomyopathy, a coronary artery disease, a left ventricular non-compaction cardiomyopathy, a restrictive cardiomyopathy, a cardiac amyloidosis, a hypertensive heart disease, a myocarditis, an arrhythmogenic right ventricular cardiomyopathy, a pulmonary arterial hypertension, or a congenital heart disease.
